(19) United States
(12) Patent Application Publication
(10) Pub. No.: US 2015/0269307 A1
(43) Pub. Date: Sep. 24, 2015

(54) TARGET INHIBITION MAP SYSTEM FOR COMBINATION THERAPY DESIGN AND METHODS OF USING SAME

(71) Applicant: Texas Tech University System, Lubbock, TX (US)

(72) Inventors: Ranadip Pal, Lubbock, TX (US); Charles Keller, III, Portland, OR (US); Noah E. Berlow, Lubbock, TX (US); Bernard Seguin, Fort Collins, CO (US)

(21) Appl. No.: 14/661,918

(22) Filed: Mar. 18, 2015

Related U.S. Application Data
(60) Provisional application No. 61/955,068, filed on Mar. 18, 2014.

(51) Int. Cl. G06F 19/12; G06F 19/24; C12Q 1/68; G01N 33/574; G01N 33/50

(52) U.S. Cl. CPC G06F 19/12; G01N 33/574; G01N 33/5011; C12Q 1/6886; G06F 19/24; G01N 2500/02; G01N 2500/10; C12Q 2600/136; G01N 2570/00; C12Q 2600/106

(57) ABSTRACT

Disclosed is a system and method for targeted therapy designs which model a cancer pathway for predicting the effectiveness of targeted anti-cancer drugs. The disclosed system and method includes utilization of a computer processor allowing for the selection of a set of drugs from available drugs using approximation algorithms utilizing cell viability data associated with a testable culture of a patient's tumor for generating a probabilistic target inhibition map (PTIM) from viability data for considering the selection of a set of drugs from available drugs, and further supports a wide variety of scenarios for personalized cancer therapy, related products and services.

### CROSS-REFERENCE TO RELATED APPLICATIONS

The present application is related to U.S. Provisional Patent Application 61/955,068 filed on Mar. 18, 2014, titled “Target Inhibition Map System For Combination Therapy Design and Methods of Using Same,” the content of which is incorporated by reference in its entirety.

### STATEMENT REGARDING FEDERALLY SPONSORED RESEARCH OR DEVELOPMENT

This invention was made with Government support under Grant (Contract) No: CCF0953366 awarded by the National Science Foundation.

This application includes material that is subject to copyright protection. The copyright owner has no objection to the facsimile reproduction by anyone of the patent disclosure, as it appears in the Patent and Trademark Office files or records, but otherwise reserves all copyright rights whatsoever.

### TECHNICAL FIELD

The present disclosure relates in general to the field of drug sensitivity prediction. In particular, the system provides for targeted therapy designs which model a cancer pathway for predicting the effectiveness of targeted anti-cancer drugs. The disclosed systems and methods support a wide variety of scenarios for personalized cancer therapy and related products and services.

### BACKGROUND OF THE DISCLOSURE

Drugs that target specific proteins, e.g. kinases, are becoming common in cancer research. Cancer related kinases are the paradigm of molecularly-targeted therapies, and a cornerstone of personalized cancer therapy. The kinome consists of greater than 500 diverse tyrosine- or serine-threonine kinases. Some of these drugs have the capacity to target multiple kinases and effects of multiple kinase inhibition are still to be properly understood.

The structure of kinases has been well studied for increasing the specificity of targeted drugs or high throughput kinase profiling has been used as a platform for drug discovery. However, approaches for high throughput analysis of multiple kinase inhibitor data to predict the behavior of new drugs are lacking in the literature. Prediction of tumor sensitivity to drugs has been approached earlier as a classification problem using gene expression profiles in Staunton J E, et al: Chemosensitivity prediction by transcriptional profiling. Proc Natl Acad Sci 2001, 98:10787-10792.

The success of targeted anti-cancer drugs is frequently hindered by the lack of knowledge of the individual pathway of the patient and the extreme data requirements on the estimation of the personalized genetic network of the patient's tumor. The prediction of tumor sensitivity to targeted drugs remains a major challenge in the design of optimal therapeutic strategies. The current sensitivity prediction approaches are primarily based on genetic characterizations of the tumor sample.

In the last decade, a number of drugs targeting specific biologically relevant kinases have been developed that are becoming common in cancer research as a basis for personalized therapy. The idea of treating cancer through inhibition of a specific tyrosine kinase was proven by the discovery that patients with Chronic Myeloid Leukemia can be successfully treated by inhibiting the tyrosine kinase BCR-ABL with the kinase inhibitor imatinib mesylate. See Druker B J: Translation of the Philadelphia chromosome into therapy for CML. Blood 2008, 112(13):4808-17. However, the success rate of any one specific targeted drug for other forms of cancer, such as sarcoma, is limited as the tumors exhibit a wide variety of signaling pathways and are not uniformly dependent on the activity of a specific kinase.

The numerous aberrations in molecular pathways that can produce cancer are one cause to necessitate the use of drug combinations for treatment of individual cancers. Combination therapy design requires a framework for inference of the individual tumor pathways, prediction of tumor sensitivity to targeted drug(s) and algorithms for selection of the drug combinations under different constraints. The current state of the art in predicting sensitivity to drugs is primarily based on assays measuring gene expression, protein abundance and genetic mutations of tumors; these methods often have low accuracy due to the breadth of available expression data coupled with the absence of information on the functional importance of many genetic mutations. A commonly used method for predicting the success of targeted drugs for a tumor sample is based on the genetic aberrations in the tumor (e.g. mutation, amplification). However, the accuracy of prediction of drug sensitivity based on mutation knowledge is limited in many forms of tumors as some of the mutations (or low frequency polymorphisms) may not be functionally important or tumors can develop without the known genetic mutations. Statistical tests have been used in Sos M L, et al: Predicting drug susceptibility of non-small cell lung cancers based on genetic lesions. J Clin Invest 2009, 119(6):1727-1740 to show that genetic mutations can be predictive of the drug sensitivity in non-small cell lung cancers but the classification rates of these predictors based on individual mutations for the aberrant samples are still low. For specific diseases, some mutations have been able to predict the patients that will not respond to particular therapies: for instance Molinari F, Felicioni L, Buscarino M, De Dosso S, Buttitta F, Malatesta S, Movilia A, Luoni M, Boldorini R, Alabiso O, Girlando S, Soini B, Spitale A, Di Nicolantonio F, Saletti P, Crippa S, Mazzucchelli L, Marchetti A, Bardelli A, Frattini M: Increased detection sensitivity for KRAS mutations enhances the prediction of anti-EGFR monoclonal antibody resistance in metastatic colorectal cancer. Clin Cancer Res 2011, 17(14):4901-4914, reports a success rate of 87% in predicting non-responders to anti-EGFR monoclonal antibodies using the mutational status of KRAS, BRAF, PIK3CA and PTEN. The prediction of tumor sensitivity to drugs has also been approached as a classification problem using gene expression profiles. In Staunton et al., gene expression profiles are used to predict the binarized efficacy of a drug over a cell line with the accuracy of the designed classifiers ranging from 64% to 92%. In Lee J K, Havaleshko D M, Cho H, Weinstein J N, Kaldjian E P, Karpovich J, Grimshaw A, Theodorescu D: A strategy for predicting the chemosensitivity of human cancers and its application to drug discovery. Proc Natl Acad Sci 2007, 104(32):13086-13091, a co-expression extrapolation (COXEN) approach is used to predict the binarized drug sensitivity in data points outside the training set with an accuracy of around 75%. In Riddick G, Song H, Ahn S, Walling J, Borges-Rivera D, Zhang W, Fine H A: Predicting in vitro drug sensitivity using random forests. Bioinformatics 2011, 27(2):220-224, a Random Forest based ensemble approach was used for prediction of drug sensitivity and achieved an R2 value of 0.39 between the predicted IC50s and experimental IC50s. Supervised machine learning approaches using genomic signatures achieved a specificity and sensitivity of higher than 70% for prediction of drug response in Venkatesan K, Stransky N, Margolin A, Reddy A, Raman P, Sonkin D, Jones M, Wilson C, Kim S, Warmuth M, Sellers W, Lehar J, Barretina J, Caponigro G, Garraway L, Morrissey M: Prediction of drug response using genomic signatures from the Cancer Cell Line Encyclopedia. AACR Meet Abstr 2010, 2010:PR2. http://www.aacrmectingabstracts.org. Tumor sensitivity prediction has also been considered as (a) a drug-induced topology alteration. See, Mitsos A, Melas I N, Siminelakis P, Chairakaki A D, Saez-Rodriguez J, Alexopoulos L G: Identifying drug effects via pathway alterations using an integer linear programming optimization formulation on phosphoproteomic data. PLoS Comput Biol 2009, 5(12):e1000591+ using phosphoproteomic signals and prior biological knowledge of a generic pathway and (b) a molecular tumor profile based prediction. See, Sos M L, et al; and Walther Z, Sklar J: Molecular tumor profiling for prediction of response to anticancer therapies. Cancer J 2011, 17(2):71-9.

Recent literature has shifted away from the gene expression profiles in favor of utilizing the kinase inhibitor profiles of drugs. See Pal R, Berlow N: A Kinase inhibition map approach for tumor sensitivity prediction and combination therapy design for targeted drugs. Pacific Symposium on Biocomputing 2012:351-62, incorporated herein in its entirety. The kinase inhibitor profiles of a drug are not dependent on a cell line and thus for a new cell line corresponding to a patient, it is not necessary measure the gene expression profiles; rather the tumor sensitivity is measured after application of different drugs. Furthermore, a stochastic approach can predict the effectiveness of a drug on more than binary levels and produce higher accuracy results.

Drugs like staurosporine have numerous kinase targets and are extremely effective in inhibiting the tumor but also have high toxicity. In fact, the toxicity of staurosporine is similar to chemotherapy and thus working against the goal of targeted action on tumor cells without damaging normal cells and tissues. On the other hand, imatinib (Gleevec) that has minimal kinase targets are being taken by leukemia patients for over a decade with few-to-no major side effects.

Thus, it is extremely important that the off-target effects of drugs are taken into consideration before prescribing a therapeutic regime for a tumor patient. Having a mathematical framework for the possible tumor pathways can generate the minimal set of kinase inhibition combinations that are required to block all the activated tumor pathways. A combination of drugs can be selected that just targets a minimal set of kinase inhibitors, with extremely few off target inhibitions. A majority of the current approaches for modeling genetic regulatory networks are not well suited for tackling this issue as the data requirements for model parameter estimation are significantly more in terms of number of samples and requirement of primarily time series data for estimation of the model parameters. As such, advancements are necessary to utilize steady-state experimental data on drug sensitivities over cell lines or animal models to generate robust maps for representing the tumor pathways. There are currently no available sensitivity prediction approaches based on functional perturbation data that incorporates the drug protein interaction information and sensitivities to a training set of drugs with known targets.

### SUMMARY OF THE DISCLOSURE

The present disclosure addresses the limitations of the art by providing: (1) methods for extraction of numerically relevant drug targets from single-run drug screens, (2) design of the personalized Probablistic Target Inhibition Map (PTIM or TIM) circuit based on drug perturbation data, (3) algorithms for sensitivity prediction of a new drug or drug cocktail, additional algorithms and methods for combining PTIMs with data generated from secondary screens to develop a genomics and proteomics informed PTIM (GPI-PTIM), (4) validation over canine osteosarcoma, murine rhabdomyosarcoma, murine diffuse intrinsic pontine glioma (DIPG), and human DIPG primary tumors and (5) pathway flow inference using sequential protein expression measurements. The present disclosure utilizes personalized PTIMs and GPI-PTIMs to study enzyme proteins (such as kinases, histone deacetylases) as well as non-enzymes (p53). An exemplary embodiment of the present disclosure is shown in FIG. 1.

The perturbation data required for the present disclosure originates from a drug screen consisting of 60 small molecule inhibitors with quantified kinase interaction behaviors. This drug screen, denoted Drug Screen Version 1.0, consists of two sets of data: (i) the first set is the experimentally generated drug sensitivities provided as 50% growth inhibitory concentration (IC50) values. The IC50 values denote the amount of a drug required to reduce the viable population of cancerous cells in vitro by half. The sensitivity values are expected to change during each new cell line/tumor culture experiment. The generation of the sensitivities in step C can be done within 72 hours of initial biopsy using drug sensitivity assays which is a period of limited cell divisions for most primary cultures. Thus, the estimated personalized maps may be closer to real-time circuits in cancer cells—akin to the signaling found in an untreated patient within a day or two after biopsy, and not the evolving consensus pattern of signaling for growing and dividing tumor cells as subpopulations emerge with increased fitness in vitro. (ii) In addition, the drug screen contains experimentally derived half-maximal concentration (EC50) values for the interaction of each drug and each kinase (enzyme) target. The EC50 value is directly related to the notion of inhibition of a kinase target; in particular, the EC50 values correspond to the amount of a compound needed to deactivate 50% of the enzyme (eg, kinase) activity of the associated target protein. Hence, for a drug compound, a target with a lower EC50 is the one that will be efficiently/potently inhibited at low drug concentration levels. Thus, low EC50 targets are often considered to be the primary targets of a drug. The remaining targets are considered to be the side targets of a drug, and are often ignored. The utility of this EC50 data is its consistency throughout experiments; the EC50 values as curated from literature searches are fixed, regardless of change of tumor type or patient of origin. This provides a great amount of prior information for analysis of the drug screen results

The present disclosure provides an input-output mathematical framework for the analysis of and inference on the functional, genomic and proteomic data generated by the drug screens for the purpose of anti-cancer drug sensitivity prediction and inference of personalized tumor survival pathway. The present disclosure may be performed by a computer or device as set forth herein. The personalized tumor survival pathway refers to the visual circuit diagram generated from the inferred Probabilistic Target Inhibition Map as (PTIM). Note that the circuit corresponding to a PTIM is only a coarse representation of the PTIM for visual understanding of the most probable target combinations whose inhibition can reduce the tumor survival. Since the experiments were conducted on in-vitro cell cultures with the output being cell viability measured in terms of IC50, the survival here refers to tumor cell culture survival and not the overall survival of the patient.

In one aspect, a method for designing a combination drug cancer therapy is provided. In some embodiments, the method comprises (a) identifying a patient diagnosed with a cancer, (b) generating a testable culture from the cancer, (c) testing viability of the testable culture against one or more targeted drugs, (d) generating, via a computing device, a probabilistic target inhibition map (PTIM) from the viability data that produces the lowest error in sensitivity prediction; wherein the computational algorithm involves (i) a selection of a set of targets that satisfies the biological constraint of increased inhibition of oncogenic targets increasing sensitivity, and (ii) generation of a probabilistic model based on the selected targets that produces high accuracy sensitivity prediction for unknown drugs with known target inhibition profile, (e) generating data from one or more secondary screens of the cancer or the testable culture, (f) creating, via a computing device, a genomics and proteomics informed PTIM (GPI-PTIM) based on the PTIM and the data from step (e), (g) designing, via a computing device, a designed combination therapy based on the GPI-PTIM, wherein the GPI-PTIM is configured to yield higher sensitivity with minimal target inhibition or avoiding resistance to drugs by targeting multiple pathways, and (h) validating the designed combination therapy in vitro against the testable culture to yield a validated combination therapy. In some embodiments, the designing step (g) further comprises considering the selection of a set of drugs from available drugs using approximation algorithms, wherein the combined toxicity of the set of drugs is restricted by an upper bound and the sensitivity of the combination drugs is predicted to be synergistic. In some embodiments, the method includes the additional step (j) treating the patient with the validated combination therapy.

In one aspect, the method further comprises validating the combination therapy using an in vivo model, which may be an animal model, a mouse model, and specifically may be a mouse xenograft model. In some embodiments, the validated combination therapy will be selected from the designed combination therapy that demonstrates the best activity against the patient's cancer in both the in vitro and in vivo testing models.

In another aspect, the method will further comprise repeating steps (c) to (h) in the event that a validated combination therapy is not identified. In some embodiments, the method will further comprise repeating steps (b) to (h) in the event that a validated combination therapy is not identified.

In other aspects, the secondary screens of the method may be any or all of RNA sequencing, DNA sequencing, protein expression testing, histomorphology, or medical imaging. In some embodiments, the protein expression testing may comprise immunohistochemistry scoring. In some embodiments the histomorphology may comprise round versus spindle cell feature scoring. In some embodiments the medical imaging may comprise imaging cellular features such as shape, roundness, or the interdigitating roughness of an invasive tumor. In another aspect the testable culture may be a single cell suspension, a primary cell culture, or a cell line established from the cancer of the patient.

Another aspect disclosed is a method of using a computing device to design and communicate over a network to a user a designed combination therapy that may be validated. In some embodiments, the method comprises (a) receiving, at a computing device over a network from a user, a cell viability query comprising cell viability data associated with a testable culture of a patient's tumor; (b) extracting, via the computing device, features of the cell viability data from the testable culture, said features comprising information associated with one or more targeted drugs; (c) generating, via the computing device, a probabilistic target inhibition map (PTIM) from the viability data that produces the lowest error in sensitivity prediction; wherein the computational algorithm involves (i) a selection of a set of targets that satisfies the biological constraint of increased inhibition of oncogenic targets increasing sensitivity, and (ii) generation of a probabilistic model based on the selected targets that produces high accuracy sensitivity prediction for unknown drugs with known target inhibition profile; (d) receiving, at a computing device, secondary data from one or more cancer screens from the testable culture; (e) comparing, via the computing device, the PTIM of the cell viability data with the secondary data from one or more secondary screens from the testable culture, said comparison comprising creating a genomics and proteomics informed PTIM (GPI-PTIM); (f) designing, via the computing device, a designed combination therapy based on the GPI-PTIM, wherein the GPI-PTIM is configured to yield higher sensitivity with minimal target inhibition or avoiding resistance to drugs by targeting multiple pathways; and (f) communicating, via the computing device over the network, a designed combination therapy that may be validated to a validated combination therapy.

In another aspect, the designing step (f) further comprises considering the selection of a set of drugs from available drugs using approximation algorithms via the computing device, wherein the combined toxicity of the set of drugs is restricted by an upper bound and the sensitivity of the combination drugs is predicted to be synergistic. In some embodiments, the method further comprises a new step (g) which entails repeating steps (a) to (f) if a validated combination therapy is not identified.

Another aspect disclosed is a system for designing a combination therapy. In some embodiments, the system comprises a process and a non-transitory computer-readable storage medium for tangibly storing thereon program logic for execution by the processor. In some embodiments, the program logic comprises (i) receiving logic executed by the processor for receiving, over a network from a user, a cell viability query comprising cell viability data associated with a testable culture of a patient's tumor; (ii) extracting logic executed by the processor for extracting features of the cell viability data from the testable culture, said features comprising information associated with one or more targeted drugs; (iii) generating logic executed by the processor for generating a probabilistic target inhibition map (PTIM) from the viability data that produces the lowest error in sensitivity prediction; wherein the computational algorithm involves (i) a selection of a set of targets that satisfies the biological constraint of increased inhibition of oncogenic targets increasing sensitivity, and (ii) generation of a probabilistic model based on the selected targets that produces high accuracy sensitivity prediction for unknown drugs with known target inhibition profile; (iv) receiving logic executed by the processor for receiving secondary data from one or more secondary screens from the testable culture; (v) comparing logic executed by the processor for comparing the PTIM of the cell viability data with the secondary data from one or more cancer screens from the testable culture, said comparison comprising creating a genomics and proteomics informed PTIM (GPI-PTIM); (vi) designing logic executed by the processor for designing a designed combination therapy based on the GPI-PTIM, wherein the GPI-PTIM is configured to yield higher sensitivity with minimal target inhibition or avoiding resistance to drugs by targeting multiple pathways; and (vi) communicating logic executed by the processor for communicating, over the network a designed combination therapy. In some embodiments the designing logic further comprises logic for considering the selection of a set of drugs from available drugs using approximation algorithms, wherein the combined toxicity of the set of drugs is restricted by an upper bound and the sensitivity of the combination drugs is predicted to be synergistic.

### BRIEF DESCRIPTION OF THE DRAWINGS

The foregoing and other objects, features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 depicts one combination therapy workflow of the present disclosure.

FIG. 2 depicts a second combination therapy workflow of the present disclosure.

FIG. 3 depicts a PTIM circuit for an osteosarcoma primary culture.

FIG. 4 depicts a schematic representation of an experimental and computational approach to targeted therapy combination predictions.

FIG. 5 depicts a Venn diagram of chemical screen hits, RNA sequencing, siRNA screening data and phosphoproteomic analyses.

FIG. 6 depicts proteomic based target inhibition maps for U23674 aRMS.

FIG. 7 depicts visualizations of pathways implicated in one variation of a PTIM model.

FIG. 8 depicts combination screening results and combination index values for one drug combination.

FIG. 9 depicts one model of a chemical screen hit analysis.

FIG. 10 depicts cellular viability of a testable culture following siRNA RAPID screen assay.

FIG. 11 depicts samples, methods and results for undifferentiated pleomorphic sarcoma in a PTIM-model guided abrogation of resistance experiment.

FIG. 12 depicts a low dose combination screen result for one drug combination.

FIG. 13 depicts the PTIM model of a xenograft-derived chemical screen.

FIG. 14 depicts the mean tumor volume of an in vivo testing of a PTIM model.

FIG. 15 depicts computational modeling to identify salient gene targets.

FIG. 16 depicts the validation of predicted synergy of PI3K and mTOR inhibition.

FIG. 17 is a block diagram illustrating architecture of a hardware device in accordance with one or more embodiments of the present disclosure.

FIG. 18 is a schematic block diagram illustrating components of a system in accordance with embodiments of the present disclosure.

FIG. 19 is a block diagram illustrating architecture of a hardware device in accordance with one or more embodiments of the present disclosure.

### DETAILED DESCRIPTION OF THE DISCLOSURE

While the making and using of various embodiments of the present disclosure are discussed in detail below, it should be appreciated that the present disclosure provides many applicable inventive concepts that can be embodied in a wide variety of specific contexts, goods, or services. The specific embodiments discussed herein are merely illustrative of specific ways to make and use the disclosure and do not delimit the scope of the disclosure.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The present disclosure will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used.

Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. Computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

The principles discussed herein may be embodied in many different forms. The preferred embodiments of the present disclosure will now be described, and shall include the embodiments set forth in hereto.

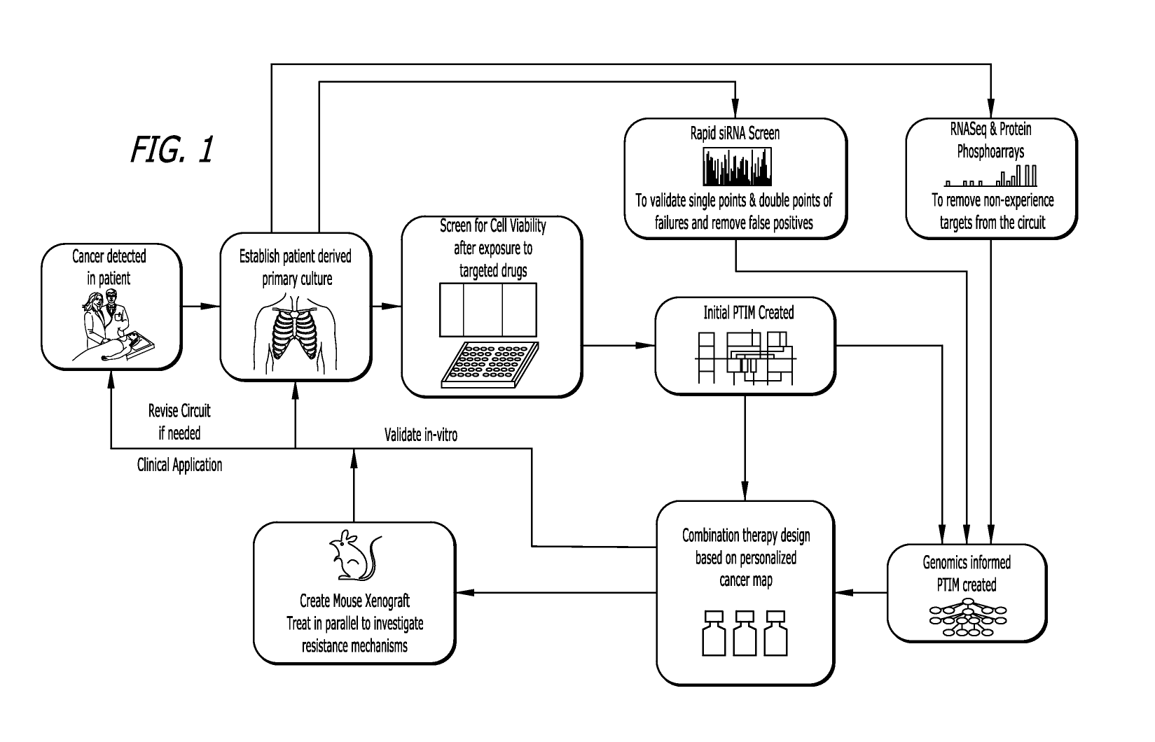

FIG. 1 explains the various steps of one embodiment of the method as follows: A patient is diagnosed with cancer, and identified as a patient for which a personalized, combination therapy can be generated. Initially, a testable culture of the tumor is established. In some embodiments this testable culture may be a single cell analysis, in other embodiments it can be a primary culture, a cell line, or any culture of cells derived from the primary tumor that may be analysed by the subsequent steps of the method. Cell viability after exposure to targeted drugs is measured through a drug screen. Use of this functional data rather than mutation or protein biomarkers provides a unique advantage. A probabilistic target inhibition map (PTIM) is then generated based on the IC₅₀'s and the known targets of the drugs in the screen. PTIM denotes a predictive model that provides the sensitivity for all possible target inhibitions. Specifically, a PTIM is composed of a set T={T₁, T₂, . . . , Tₙ} consisting of binary variables, each denoting inhibition of a target, and a function ƒ relating the target inhibitions to the steady state sensitivity y_{T}, i.e. y_{T}=(T₁, T₂, . . . , Tₙ). The inhibition vector (e₁, e₂, . . . , eₙ) corresponding to a drug is known as the Drug Target Inhibition Profile (DTIP). The coarse structure of the PTIM can also be represented by an abstract pathway which will be termed PTIM Circuit. The construction of the PTIM Circuit is explained in the methods section. Next, further data is collected using secondary screens which may be RNA sequencing and Protein phosphoarrays (which remove non-experience targets from the circuit) and rapid siRNA screens (to validate single points of failures and remove false positives). Based on the knowledge of the PTIM and PTIM-directed optimized protein expression measurements (model based experimentation), a genomics and proteomics informed PTIM model is generated (which may also be referred to as a dynamic model as seen in FIG. 1). The dynamic model yields a combination therapy designed through the utilization of the personalized PTIM (also called “cancer map” as seen in FIG. 1) and the dynamic model (if estimated). Various constraints such as avoiding resistance to drugs or minimizing toxicity can be applied to design the combination therapy. Concurrently, or separately, the designed combination therapy may be validated in any one of a number of in vivo and in vitro testing models accepted by one of skill in the art. Specifically, the in vivo models may be but are not solely limited to single cell suspensions, primary tumor cultures, or cell lines generated from a patient's cancer. The generated drug combinations are validated in vitro on the primary culture, and a mouse xenograft model, as one example of an in vivo model, can be used to study development of resistance simultaneously or in addition to the in vitro testing. A validated combination therapy may be identified based on solely the in vitro data, the in vivo data, or on both the in vitro and in vivo data. If needed, the circuit is revised or the drug combination with best response in vivo (mouse) and in vitro (primary culture) is then provided to the patient.

As used herein, “testable culture” shall mean any culture of cells derived from the cancer cells of a specific patient. Such a culture may be derived from or be a single cancer cell or single cell suspension. The testable culture may be a cell culture of primary cells, it may be a cell line derived from a single cell or collection of cells removed from a patient.

As used herein, “viability” shall mean the measure of survivability of a testable culture in the presence of one or more anti-cancer compounds. This may relate to the IC50 of a drug, which is the half maximal inhibitory concentration (IC50) of the drug and is a measure of the effectiveness of a substance in inhibiting a specific biological or biochemical function. This quantitative measure indicates how much of a particular drug or other substance (inhibitor) is needed to inhibit a given biological process (or component of a process, i.e. an enzyme, cell, cell receptor or microorganism) by half. It is commonly used as a measure of antagonist drug potency in pharmacological research. The IC50 represents the concentration of a drug that is required for 50% inhibition in vitro. It is comparable to an EC50 for agonist drugs. EC50 also represents the plasma concentration required for obtaining 50% of a maximum effect in vivo. The term half maximal effective concentration (EC50) refers to the concentration of a drug, antibody or toxicant which induces a response halfway between the baseline and maximum after a specified exposure time. It is commonly used as a measure of a drug's potency. The EC50 of a graded dose response curve therefore represents the concentration of a compound where 50% of its maximal effect is observed. The EC50 of a quantal dose response curve represents the concentration of a compound where 50% of the population exhibit a response, after a specified exposure duration. It is also related to IC50 which is a measure of a compound's inhibition (50% inhibition). For competition binding assays and functional antagonist assays IC50 is the most common summary measure of the dose-response curve. For agonist/stimulator assays the most common summary measure is the EC50.

As used herein, “secondary screens” may relate to any of a number of assays or tests that can be done on the cancer cells taken from or derived from a patient that may yield information that helps in the generation of a genomics and proteomics informed probabilistic target inhibition map (GPI-PTIM). Secondary screens may be sequencing, genetic analysis, genomics, proteomics, mRNA analysis, protein expression analysis, morphological or pathology analyses such as histomorphology or spindle cell feature scoring, medical imaging which may be any form of imaging from microscoping to magnetic resonance or electron microscopy, immunohistochemistry. Medical imaging may also comprise imaging the cellular features or shape or the organization and structure of the chromosomes. It may further comprise analysing the shape, roundness or interdigitating roughness of the cancer cells.

As used herein, “combination therapy” shall mean a treatment regimen for any cancer that involves the use of two or more cancer compounds or drugs. The terms “combination therapy” or “combined treatment” or “in combination” as used herein denotes any form of concurrent or parallel treatment with at least two distinct therapeutic agents.

As used herein, the term “cancer” includes all cancers and cancer metastases, including sarcomas, carcinomas and other solid and non-solid tumor cancers. Solids cancer include but are not limited to tumors of the central nervous system, breast cancer, prostate cancer, skin cancer (including basal cell carcinoma cell carcinoma, squamous cell carcinoma and melanoma), cervical cancer, uterine cancer, lung cancer, ovarian cancer, testicular cancer, thyroid cancer, astrocytoma, glioma, pancreatic cancer, stomach cancer, liver cancer, colon cancer, renal cancer, bladder cancer, oesophageal cancer, cancer of the larynx, cancer of the parotid, cancer of the biliary tract, rectal cancer, endometrial cancer, adenocarcinomas, small cell carcinomas, neuroblastomas, mesotheliomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas, rhabdomyosarcomas, Wilms tumors, and the like. According to certain embodiments of the present invention, the cancer is selected from the group consisting of cancers of the gastrointestinal tract, pancreatic cancer and prostate cancer. Each possibility represents a separate embodiment of the present invention. According to some embodiments, the cancer of the gastrointestinal tract is selected from the group consisting of colorectal cancer and gastric cancer. According to certain embodiments, the term “cancer” further comprises pre-cancerous lesions. A small change in ligand concentration typically result in rapid changes in response in the biological system, following a sigmoidal function. The inflection point at which the increase in response with increasing ligand concentration begins to slow is the EC50, which can be mathematically determined by derivation of the best-fit line. While relying on a graph for estimation is more convenient, this typical method yields less accurate results and less precise.

The terms “treating”, “treatment” and the like are used herein to mean affecting a subject, tissue or cell to obtain a desired pharmacological and/or physiological effect. The effect may be therapeutic in terms of a partial or complete cure of the cancer. “Treating” as used herein covers any treatment of cancer in a subject; inhibiting the cancer, i.e. arresting its development; or relieving or ameliorating the effects of the cancer, i.e., cause regression of the tumor or of the effects of the cancer.

Suitable chemotherapeutic agents for use in the combinations of the present invention include, but are not limited to, alkylating agents, antibiotic agents, antimetabolic agents, hormonal agents, plant-derived agents, anti-angiogenic agents, differentiation inducing agents, cell growth arrest inducing agents, apoptosis inducing agents, cytotoxic agents, agents affecting cell bioenergetics, biologic agents, e.g., monoclonal antibodies, kinase inhibitors and inhibitors of growth factors and their receptors, gene therapy agents, cell therapy, e.g., stem cells, or any combination thereof.

Alkylating agents are drugs which impair cell function by forming covalent bonds with amino, carboxyl, sufihydryl and phosphate groups in biologically important molecules. The most important sites of alkylation are DNA, RNA and proteins. Alkylating agents depend on cell proliferation for activity but are not cell-cycle-phase-specific. Alkylating agents suitable for use in the present invention include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e. g. BCNU, carmustine, lomustine, streptozocin), nonclassic alkylating agents (e.g., altretamine, dacarbazine, and procarbazine), and platinum compounds (e.g., carboplastin, oxaliplatin and cisplatin).

Antitumor antibiotics like adriamycin intercalate DNA at guanine-cytosine and guanine-thymine sequences, resulting in spontaneous oxidation and formation of free oxygen radicals that cause strand breakage. Other antibiotic agents suitable for use in the present invention include, but are not limited to, anthracyclines (e. g. doxorubicin, daunorubicin, epirubicin, idarubicin and anthracenedione), mitomycin C, bleomycin, dactinomycin, and plicatomycin.

Antimetabolic agents suitable for use in the present invention include but are not limited to, floxuridine, fluorouracil, methotrexate, leucovorin, hydroxyurea, thioguanine, mercaptopurine, cytarabine, pentostatin, fludarabine phosphate, cladribine, asparaginase, and gemcitabine

Hormonal agents suitable for use in the present invention, include but are not limited to, an estrogen, a progestogen, an antiesterogen, an androgen, an antiandrogen, an LHRH analogue, an aromatase inhibitor, diethylstibestrol, tamoxifen, toremifene, fluoxymesterol, raloxifene, bicalutamide, nilutamide, flutamide, aminoglutethimide, tetrazole, ketoconazole, goserelin acetate, leuprolide, megestrol acetate, and mifepristone.

Plant derived agents include taxanes, which are semisynthetic derivatives of extracted precursors from the needles of yew plants. These drugs have a novel 14-member ring, the taxane. Unlike the vinca alkaloids, which cause microtubular disassembly, the taxanes (e.g., taxol) promote microtubular assembly and stability, therefore blocking the cell cycle in mitosis. Other plant derived agents include, but are not limited to, vincristine, vinblastine, vindesine, vinzolidine, vinorelbine, etoposide, teniposide, and docetaxel.

Biologic agents suitable for use in the present invention include, but are not limited to immuno-modulating proteins, monoclonal antibodies against tumor antigens, tumor suppressor genes, kinase inhibitors and inhibitors of growth factors and their receptors and cancer vaccines. For example, the immuno-modulating protein can be interleukin 2, interleukin 4, interleukin 12, interferon El interferon D, interferon alpha, erythropoietin, granulocyte-CSF, granulocyte, macrophage-CSF, bacillus Calmette-Guerin, levamisole, or octreotide. Examples of biologic agents to be used according to the teachings of the present invention are anti-CD24 antibody, cetuximab (Erbitux®) and bevacizumab (Avastin®)

Agents affecting cell bioenergetics affecting cellular ATP levels and/or molecules/activities regulating these levels

Recent developments have introduced, in addition to the traditional cytotoxic and hormonal therapies, additional therapies for the treatment of cancer. For example, many forms of gene therapy are undergoing preclinical or clinical trials. In addition, approaches based on the inhibition of tumor vascularization (angiogenesis) are currently under development. The aim of this concept is to cut off the tumor from nutrition and oxygen supply provided by a newly built tumor vascular system. In addition, cancer therapy is also being attempted by the induction of terminal differentiation of the neoplastic cells. Suitable differentiation agents include hydroxamic acids, derivatives of vitamin D and retinoic acid, steroid hormones, growth factors, tumor promoters, and inhibitors of DNA or RNA synthesis. Also, histone deacetylase inhibitors are suitable chemotherapeutic agent to be used in the present invention.

According to certain embodiments, the at least one additional anti-cancer agent is known to be effective in treating the cancer type affecting the subject. The term “anti-cancer” as used herein in reference to “anti-cancer agent”, “anti-cancer therapeutic effect” “anti-cancerous effect” and the like is meant in its broadest scope as in known in the art, and includes the activities of arrest of cell growth, induction of apoptosis, induction of differentiation, cell death and the like.

As used herein, the terms “effective amount” refers to an amount of an anti-cancer agent according to the teachings of the present invention that is effective in treating cancer as defined hereinabove. The specific “effective amount” will vary according to the particular condition being treated, the physical condition and clinical history of the subject, the duration of the treatment and the nature of the combination of agents applied and its specific formulation. As used herein, the term “therapeutically effective amount” refers to the amount of an anti-cancer agent known in the art to be effective in treating cancer cells/disease of a particular type. According to certain embodiments, the “effective amount” according to the teachings of the present invention is lower compared to the “therapeutically effective amount” as is known in the art.

Determining the dosage and duration of treatment according to any aspect of the present invention is well within the skills of a professional in the art. The skilled Artisans are readily able to monitor patients to determine whether treatment should be started, continued, discontinued or resumed at any given time. For example, dosages of the compounds are suitably determined depending on the individual cases taking symptoms, age and sex of the subject and the like into consideration. An effective amount for a particular patient may vary depending on factors such as the condition being treated, the overall health of the patient and the method, route and dose of administration. The clinician using parameters known in the art makes determination of the appropriate dose. Generally, the dose begins with an amount somewhat less than the optimum dose and it is increased by small increments thereafter until the desired or optimum effect is achieved. Suitable dosages can be determined by further taking into account relevant disclosure in the known art.

Although the components of the combination therapy of the present invention can be administered alone, it is contemplated that the components of the combination will be administered in pharmaceutical compositions further containing at least one pharmaceutically acceptable carrier or excipient. Each of the components can be administered in a separate pharmaceutical composition, or the combination can be administered in one pharmaceutical composition.

As used herein, “genomics” shall mean a discipline in genetics that applies recombinant DNA, DNA sequencing methods, and bioinformatics to sequence, assemble, and analyze the function and structure of genomes (the complete set of DNA within a single cell of an organism). Advances in genomics have triggered a revolution in discovery-based research to understand even the most complex biological systems such as the brain. The field includes efforts to determine the entire DNA sequence of organisms and fine-scale genetic mapping. The field also includes studies of intragenomic phenomena such as heterosis, epistasis, pleiotropy and other interactions between loci and alleles within the genome. In contrast, the investigation of the roles and functions of single genes is a primary focus of molecular biology or genetics and is a common topic of modern medical and biological research. Research of single genes does not fall into the definition of genomics unless the aim of this genetic, pathway, and functional information analysis is to elucidate its effect on, place in, and response to the entire genome's networks.

As used herein, “proteomics” shall mean Proteomics is the large-scale study of proteins, particularly their structures and functions. Proteins are vital parts of living organisms, as they are the main components of the physiological metabolic pathways of cells. The proteome is the entire set of proteins, produced or modified by an organism or system. This varies with time and distinct requirements, or stresses, that a cell or organism undergoes. Proteomics is an interdisciplinary domain formed on the basis of the research and development of the Human Genome Project; it is also emerging scientific research and exploration of proteomes from the overall level of intracellular protein composition, structure, and its own unique activity patterns. It is an important component of functional genomics. While proteomics generally refers to the large-scale experimental analysis of proteins, it is often specifically used for protein purification and mass spectrometry.

In one embodiment of the present invention, a system for designing a combination therapy is provided. In some embodiments, the system comprises a process and a non-transitory computer-readable storage medium for tangibly storing thereon program logic for execution by the processor. In addressing the present disclosure, computer program instructions can be provided to a processor of a general purpose computer to alter its function, a special purpose computer, ASIC, or other programmable data processing apparatus, such that the instructions, which execute via the processor of the computer or other programmable data processing apparatus, implement the functions/acts specified in the block diagrams or operational block or blocks.

For the purposes of this disclosure a “computer readable medium” (or computer-readable storage medium/media) stores computer data, which data can include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. “Computer readable storage media”, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other physical or material medium which can be used to tangibly store the desired information or data or instructions and which can be accessed by a computer or processor.

For the purposes of this disclosure the term “server” should be understood to refer to a service point, which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Servers may vary widely in configuration or capabilities, but generally a server may include one or more central processing units and memory. A server may also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network-attached storage (NAS), a storage area network (SAN), or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network. Various types of devices may, for example, be made available to provide an interoperable capability for differing architectures or protocols. As one illustrative example, a router may provide a link between otherwise separate and independent LANs.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like. Servers may vary widely in configuration or capabilities, but generally a server may include one or more central processing units and memory. A server may also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

For purposes of this disclosure, a client (or consumer or user) device may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device an Near Field Communication (NFC) device, a Personal Digital Assistant (PDA), a handheld computer, a tablet computer, a phablet, a laptop computer, a set top box, a wearable computer, an integrated or distributed device combining various features, such as features of the forgoing devices, or the like.

A client device may vary in terms of capabilities or features. Claimed subject matter is intended to cover a wide range of potential variations. For example, a smart phone, phablet or tablet may include a numeric keypad or a display of limited functionality, such as a monochrome liquid crystal display (LCD) for displaying text. In contrast, however, as another example, a web-enabled client device may include one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

A client device may include or may execute a variety of operating systems, including a personal computer operating system, such as a Windows, iOS or Linux, or a mobile operating system, such as iOS, Android, or Windows Mobile, or the like.

A client device may also include or execute an application to communicate content, such as, for example, textual content, multimedia content, or the like. A client device may also include or execute an application to perform a variety of possible tasks, such as browsing, searching, playing various forms of content, including locally stored or streamed video, or games (such as fantasy sports leagues). The foregoing is provided to illustrate that claimed subject matter is intended to include a wide range of possible features or capabilities.

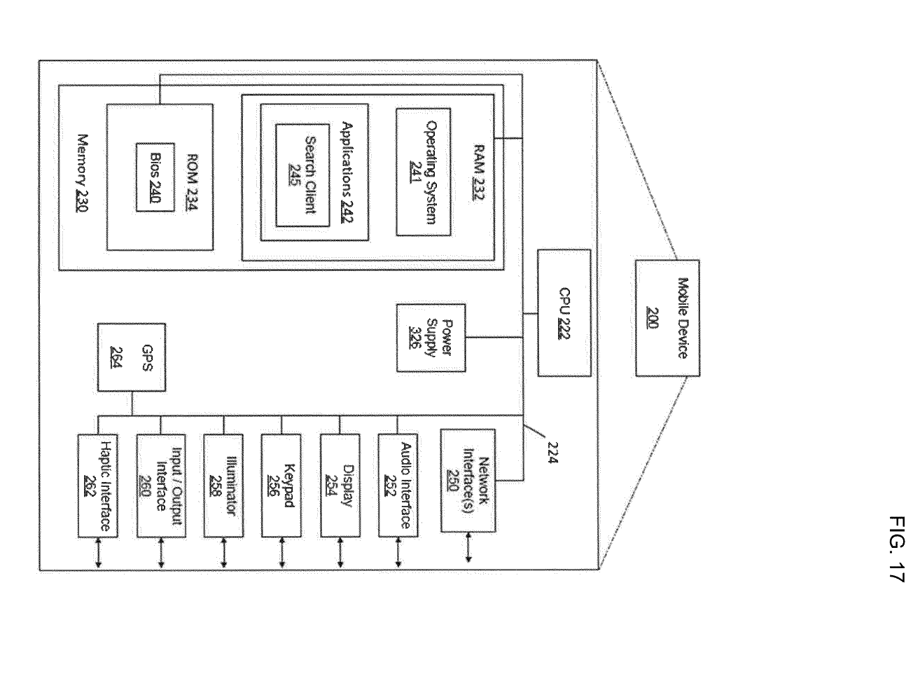

FIG. 17 is a schematic diagram illustrating a client device showing an example embodiment of a client device that may be used within the present disclosure. Client device 200 may include many more or less components than those shown in FIG. 17. However, the components shown are sufficient to disclose an illustrative embodiment for implementing the present disclosure.

As shown in the FIG. 17, client device 200 includes a processing unit (CPU) 222 in communication with a mass memory 230 via a bus 224. Client device 200 also includes a power supply 226, one or more network interfaces 250, an audio interface 252, a display 254, a keypad 256, an illuminator 258, an input/output interface 260, a haptic interface 262, and an optional global positioning systems (GPS) receiver 264. Power supply 226 provides power to Client device 200. A rechargeable or non-rechargeable battery may be used to provide power. The power may also be provided by an external power source, such as an AC adapter or a powered docking cradle that supplements and/or recharges a battery.

Client device 200 may optionally communicate with a base station (not shown), or directly with another computing device. Network interface 250 includes circuitry for coupling Client device 200 to one or more networks, and is constructed for use with one or more communication protocols and technologies including, but not limited to, global system for Client communication (GSM), code division multiple access (CDMA), time division multiple access (TDMA), user datagram protocol (UDP), transmission control protocol/Internet protocol (TCP/IP), SMS, general packet radio service (GPRS), WAP, ultra wide band (UWB), IEEE 802.16 Worldwide Interoperability for Microwave Access (WiMax), SIP/RTP, or any of a variety of other wireless communication protocols. Network interface 250 is sometimes known as a transceiver, transceiving device, or network interface card (NIC).

Audio interface 252 is arranged to produce and receive audio signals such as the sound of a human voice. For example, audio interface 252 may be coupled to a speaker and microphone (not shown) to enable telecommunication with others and/or generate an audio acknowledgement for some action. Display 254 may be a liquid crystal display (LCD), gas plasma, light emitting diode (LED), or any other type of display used with a computing device. Display 254 may also include a touch sensitive screen arranged to receive input from an object such as a stylus or a digit from a human hand.

Keypad 256 may comprise any input device arranged to receive input from a user. For example, keypad 256 may include a push button numeric dial, or a keyboard. Keypad 256 may also include command buttons that are associated with selecting and sending images. Illuminator 258 may provide a status indication and/or provide light. Illuminator 258 may remain active for specific periods of time or in response to events. For example, when illuminator 258 is active, it may backlight the buttons on keypad 256 and stay on while the client device is powered. Also, illuminator 258 may backlight these buttons in various patterns when particular actions are performed, such as dialing another client device. Illuminator 258 may also cause light sources positioned within a transparent or translucent case of the client device to illuminate in response to actions.

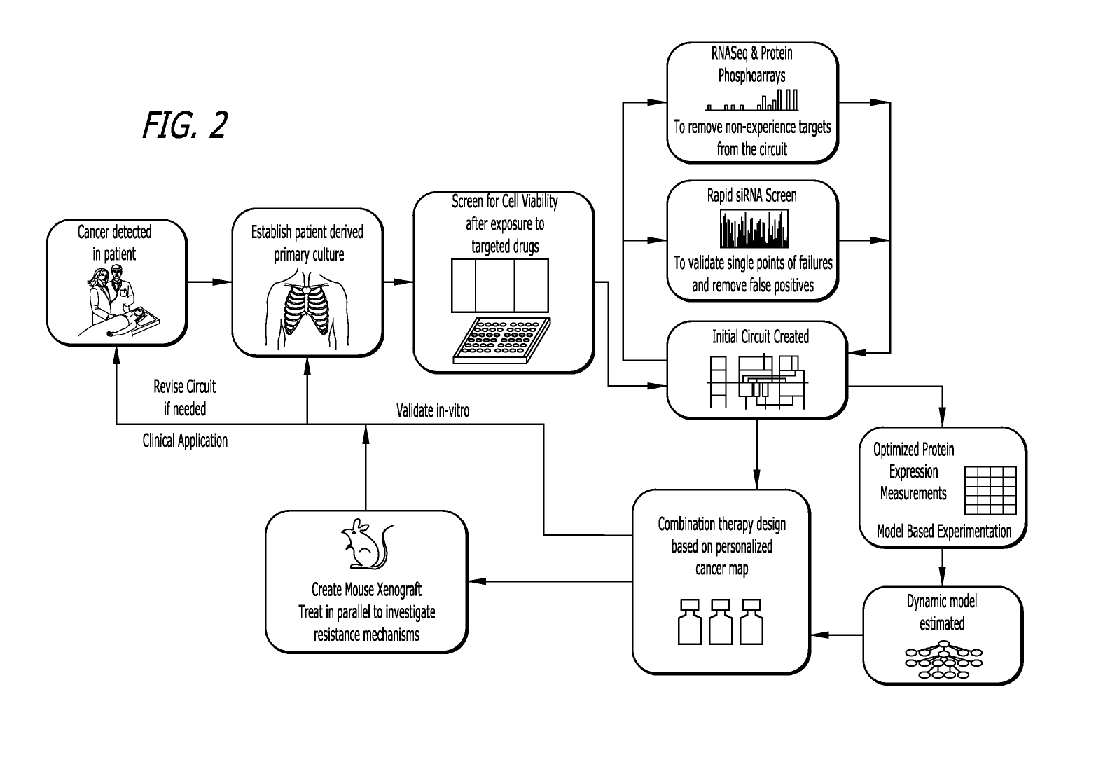

Client device 200 also comprises input/output interface 260 for communicating with external devices, such as a headset, or other input or output devices not shown in FIG. 2. Input/output interface 260 can utilize one or more communication technologies, such as USB, infrared, Bluetooth™, or the like. Haptic interface 262 is arranged to provide tactile feedback to a user of the client device. For example, the haptic interface may be employed to vibrate client device 200 in a particular way when the Client device 200 receives a communication from another user.

Optional GPS transceiver 264 can determine the physical coordinates of Client device 200 on the surface of the Earth, which typically outputs a location as latitude and longitude values. GPS transceiver 264 can also employ other geo-positioning mechanisms, including, but not limited to, triangulation, assisted GPS (AGPS), E-OTD, CI, SAI, ETA, BSS or the like, to further determine the physical location of Client device 200 on the surface of the Earth. It is understood that under different conditions, GPS transceiver 264 can determine a physical location within millimeters for Client device 200; and in other cases, the determined physical location may be less precise, such as within a meter or significantly greater distances. In one embodiment, however, Client device may through other components, provide other information that may be employed to determine a physical location of the device, including for example, a MAC address, IP address, or the like.

Mass memory 230 includes a RAM 232, a ROM 234, and other storage means. Mass memory 230 illustrates another example of computer storage media for storage of information such as computer readable instructions, data structures, program modules or other data. Mass memory 230 stores a basic input/output system (“BIOS”) 240 for controlling low-level operation of Client device 200. The mass memory also stores an operating system 241 for controlling the operation of Client device 200. It will be appreciated that this component may include a general purpose operating system such as a version of UNIX, or LINUX™, or a specialized client communication operating system such as Windows Client™, or the Symbian® operating system. The operating system may include, or interface with a Java virtual machine module that enables control of hardware components and/or operating system operations via Java application programs.

Memory 230 further includes one or more data stores, which can be utilized by Client device 200 to store, among other things, applications 242 and/or other data. For example, data stores may be employed to store information that describes various capabilities of Client device 200. The information may then be provided to another device based on any of a variety of events, including being sent as part of a header during a communication, sent upon request, or the like. At least a portion of the capability information may also be stored on a disk drive or other storage medium (not shown) within Client device 300.

Applications 242 may include computer executable instructions which, when executed by Client device 200, transmit, receive, and/or otherwise process audio, video, images, and enable telecommunication with another user of another client device. Other examples of application programs include modeling systems, PTIM modules, validation modules, task managers, transcoders, database programs, word processing programs, security applications, spreadsheet programs, games, search programs, and so forth. Applications 242 may further include search client 245 that is configured to send, to receive, and/or to otherwise process a cell viability query ult using any known or to be known communication protocols. Although a single search client 245 is illustrated it should be clear that multiple search clients may be employed. For example, one search client may be configured to enter a cell viability query, where another search client manages results, and yet another search client is configured to manage modeling systems, development of the TIM for various testing results, and the like.

Having described the components of the general architecture employed within the disclosed systems and methods, the components' general operation with respect to the disclosed systems and methods will now be described.

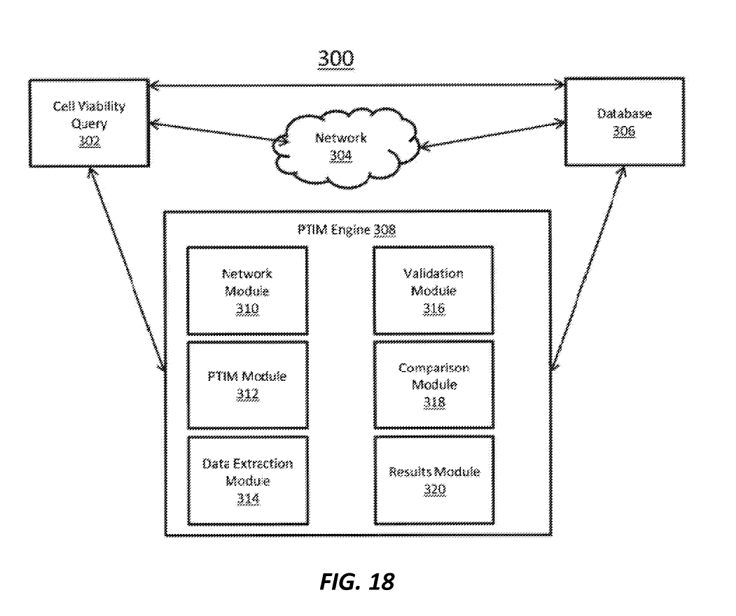

FIG. 18 is a block diagram illustrating the components of system 300 for performing the systems and methods discussed herein. FIG. 18 includes a cell viability query 302, network 304, database 306, and a PTIM engine 308. The PTIM engine 308 is a special purpose machine or processor and could be hosted by a web server, search server, content provider, user's computing device, or any combination thereof. The database 306 can be any type of database or memory. The database 306 comprises a dataset of information associated with drug sensitivity, functional and genomic information and related CCLE datasets.

The principal processor, server, or combination of devices that comprises hardware programmed in accordance with the special purpose functions herein, referred to for convenience as PTIM engine 308, includes a network module 310, PTIM module 312, data extraction module 314, validation module 316, comparison module 318 and results module 320. It should be understood that the engine(s) and modules discussed herein are non-exhaustive, as additional or fewer engines and/or modules may be applicable to the embodiments of the systems and methods discussed. The operations, configurations and functionalities of each module, and their role within embodiments of the present disclosure will be discussed with reference to FIG. 17-19. These functionalities are not simply a generic computer. Rather, the specific machines employed are highly specific to carrying out the function of the PTIM generation—as with the PTIM module, which execute the steps of the present disclosure. Specific programming in a general computer includes the PTIM module and results module for generating the data necessary for the functional aspects of the present invention. The process of the present invention may be performed by the PTIM engine 308 and focuses upon modeling PTIM data to effectuate the efficient and accurate personalized therapy approach discussed herein.

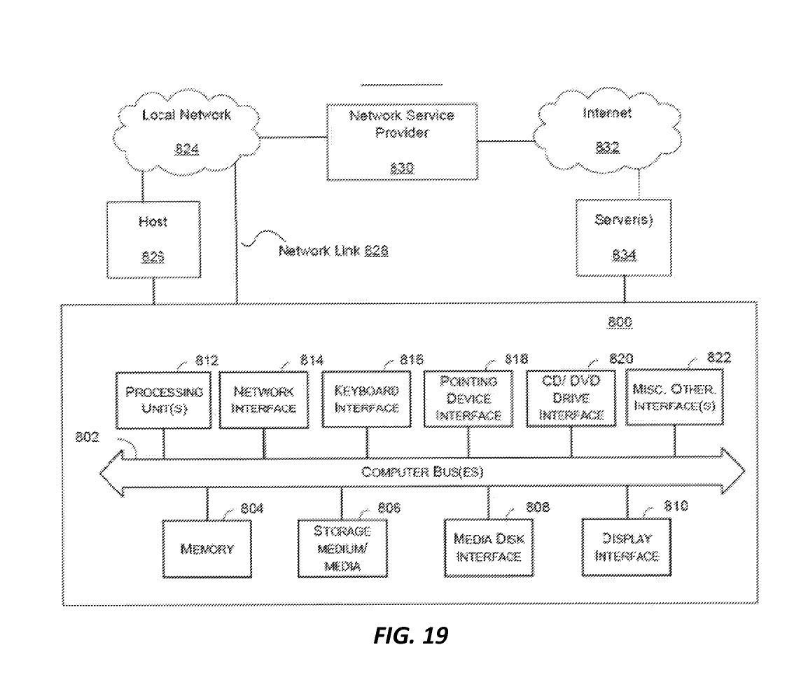

As shown in FIG. 19, internal architecture 800 of a computing device(s), computing system, computing platform and the like includes one or more processing units, processors, or processing cores, (also referred to herein as CPUs) 812, which interface with at least one computer bus 802. Also interfacing with computer bus 802 are computer-readable medium, or media, 806, network interface 814, memory 804, e.g., random access memory (RAM), run-time transient memory, read only memory (ROM), media disk drive interface 820 as an interface for a drive that can read and/or write to media including removable media such as floppy, CD-ROM, DVD, media, display interface 810 as interface for a monitor or other display device, keyboard interface 816 as interface for a keyboard, pointing device interface 818 as an interface for a mouse or other pointing device, and miscellaneous other interfaces not shown individually, such as parallel and serial port interfaces and a universal serial bus (USB) interface.

Memory 804 interfaces with computer bus 802 so as to provide information stored in memory 804 to CPU 812 during execution of software programs such as an operating system, application programs, device drivers, and software modules that comprise program code, and/or computer executable process steps, incorporating functionality described herein, e.g., one or more of process flows described herein. CPU 812 first loads computer executable process steps from storage, e.g., memory 804, computer readable storage medium/media 806, removable media drive, and/or other storage device. CPU 812 can then execute the stored process steps in order to execute the loaded computer-executable process steps. Stored data, e.g., data stored by a storage device, can be accessed by CPU 812 during the execution of computer-executable process steps.

Persistent storage, e.g., medium/media 806, can be used to store an operating system and one or more application programs. Persistent storage can also be used to store device drivers, such as one or more of a digital camera driver, monitor driver, printer driver, scanner driver, or other device drivers, web pages, content files, playlists and other files. Persistent storage can further include program modules and data files used to implement one or more embodiments of the present disclosure, e.g., listing selection module(s), targeting information collection module(s), and listing notification module(s), the functionality and use of which in the implementation of the present disclosure are discussed in detail herein.

Network link 828 typically provides information communication using transmission media through one or more networks to other devices that use or process the information. For example, network link 828 may provide a connection through local network 824 to a host computer 826 or to equipment operated by a Network or Internet Service Provider (ISP) 830. ISP equipment in turn provides data communication services through the public, worldwide packet-switching communication network of networks now commonly referred to as the Internet 832.

A computer called a server host 834 connected to the Internet 832 hosts a process that provides a service in response to information received over the Internet 832. For example, server host 834 hosts a process that provides information representing video data for presentation at display 810. It is contemplated that the components of system 800 can be deployed in various configurations within other computer systems, e.g., host and server.

At least some embodiments of the present disclosure are related to the use of computer system 800 for implementing some or all of the techniques described herein. According to one embodiment, those techniques are performed by computer system 800 in response to processing unit 812 executing one or more sequences of one or more processor instructions contained in memory 804. Such instructions, also called computer instructions, software and program code, may be read into memory 804 from another computer-readable medium 806 such as storage device or network link. Execution of the sequences of instructions contained in memory 804 causes processing unit 812 to perform one or more of the method steps described herein. In alternative embodiments, hardware, such as ASIC, may be used in place of or in combination with software. Thus, embodiments of the present disclosure are not limited to any specific combination of hardware and software, unless otherwise explicitly stated herein.

The signals transmitted over network link and other networks through communications interface, carry information to and from computer system 800. Computer system 800 can send and receive information, including program code, through the networks, among others, through network link and communications interface. In an example using the Internet, a server host transmits program code for a particular application, requested by a message sent from computer, through Internet, ISP equipment, local network and communications interface. The received code may be executed by processor 802 as it is received, or may be stored in memory 804 or in storage device or other non-volatile storage for later execution, or both.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

Thus, in an exemplary embodiment of the present disclosure, a system is provided for integrating a computing device having a processor which allows for the selection of a set of drugs from available drugs using approximation algorithms, wherein the combined toxicity of the set of drugs is restricted by an upper bound and the higher sensitivity is predicted to be synergistic; and a non-transitory computer-readable storage medium for tangibly storing thereon program logic for execution by the processor, the program logic comprising receiving logic executed by the processor for receiving, over a network from a user, a cell viability query comprising cell viability data associated with a testable culture of a patient's tumor; extracting logic executed by the processor for extracting features of the cell viability data from the testable culture, said features comprising information associated with one or more targeted drugs; generating logic executed by the processor for generating a probabilistic target inhibition map (PTIM) from the viability data that produces the lowest error in sensitivity prediction; wherein the computational algorithm involves (i) a selection of a set of targets that satisfies the biological constraint of increased inhibition of oncogenic targets increasing sensitivity, and (ii) generation of a probabilistic model based on the selected targets that produces high accuracy sensitivity prediction for unknown drugs with known target inhibition profile; receiving logic executed by the processor for receiving secondary data from one or more secondary screens from the testable culture; comparing logic executed by the processor for comparing the PTIM of the cell viability data with the secondary data from one or more cancer screens from the testable culture, said comparison comprising creating a genomics and proteomics informed PTIM (GPI-PTIM); designing logic executed by the processor for designing a designed combination therapy based on the GPI-PTIM, wherein the GPI-PTIM is configured to yield higher sensitivity with minimal target inhibition or avoiding resistance to drugs by targeting multiple pathways; and communicating logic executed by the processor for communicating, over the network a designed combination therapy. The designing logic further comprises logic for considering the selection of a set of drugs from available drugs using approximation algorithms, wherein the combined toxicity of the set of drugs is restricted by an upper bound and the higher sensitivity is predicted to be synergistic.

Those skilled in the art will recognize that the methods and systems of the present disclosure may be implemented in many manners and as such are not to be limited by the foregoing exemplary embodiments and following examples. Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

### EXAMPLES

The examples below provide illustrative embodiments of the present disclosure. While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

### Example 1

### Pre-Clinical Evaluation of BMS-754807, an IGF-1R Inhibitor in a Genetically Engineered Diffuse Intrinsic Pontine Glioma Mouse Model

Diffuse intrinsic pontine gliomas (DIPGs), which arise at a median age of 6-7 years old, represent a particularly lethal type of brain cancer with no effective therapeutic options. It has been previously reported the development of a DIPG mouse model which closely mirrors the human condition using the RCAS/tv-a system. This model serves as an accurate platform in which to test novel therapeutics prior to the initiation of costly human clinical trials. In this example, an in vitro high throughput drug screen as part of the DIPG preclinical consortium identified BMS-754807 as a drug of interest in DIPG. BMS-754807 is a potent and reversible small molecule tyrosine kinase inhibitor of the IGF-1R. In vitro evaluation showed significant cytotoxic effects at clinically achievable concentrations with an IC₅₀ of 0.19 μM, and significant inhibition of proliferation at concentrations of 0.1 μM. BMS-754807 inhibited IGF-1R and AKT activation in a dose dependent manner. In vivo, systemic administration of BMS 754807 to DIPG-bearing mice significantly induced apoptosis as measured by cleaved caspase 3. Together, these results suggest that BMS-754807 should be evaluated in clinical trials for children with DIPG.

### INTRODUCTION

An estimated 4,000 new malignant and non-malignant brain tumors are diagnosed annually in children in the United States [1,2]. Up to 20% of malignant central nervous system (CNS) tumors in children arise in the brainstem, with the majority being the diffuse intrinsic pontine glioma (DIPG) subtype [1,3]. DIPG is a high-grade glioma (HGG) that originates in the pons and is seen almost exclusively in children with a median age of diagnosis of 6 to 7 years [4-6].

Despite numerous clinical investigations to date, there have been no chemotherapeutic or biological agents that have proven clearly beneficial for the treatment of DIPG. The standard treatment remains conventional focal radiotherapy (RT) with a treatment dose of 54-60Gy, fractionated over a 6-week period [7]. This treatment has been shown to transiently improve neurologic function or temporarily stabilize disease in 70% of patients, but the effect on overall survival is minimal with more than 90% of children dying within two years of diagnosis [5-8].

A key to improving these outcomes is to gain a better understanding of DIPG tumor biology. In France, biopsies have been routinely performed since 2003 for both atypical and typical DIPG evaluation, and have been associated with minimal morbidity and high diagnostic yield [9]. This surgical success has also led to an increase in available DIPG tumor samples, providing much of the new biologic, molecular and genetic data that is currently advancing our understanding of DIPG biology. The most common genetic alterations include a K27M mutation in H3.3 or H3.1, which are found in up to 78% of DIPGs while p53 mutations are found in up to 77% [10,11]. Other recently discovered alterations include amplification of components of the Receptor Tyrosine Kinase-Ras-PI3K signaling pathway, with gains in platelet-derived growth factor receptor (PDGFR-A) as the most commonly amplified component [12-15].

In the United States, it remains uncommon for children with typical DIPGs to undergo stereotactic biopsy and many families elect to forgo autopsies, making human samples exceedingly rare for pre-clinical targeted drug evaluation [16]. Alternately, genetically engineered DIPG preclinical animal models have been developed with histologic and immunophenotypic similarities to human DIPG samples, and can help prioritize the translation of novel agents into clinical trials for children with DIPG [17,18]. As part of a combined effort of the Children's Oncology Group CNS-DVL committee preclinical consortium, in this study, mouse model derived DIPG tumor cells were used in a high throughput drug screen to identify novel therapeutic agents with future potential to enter clinical trials. Through this screen, IGF-1R was identified as a target of interest in DIPG.

The insulin like growth factor-1 receptor (IGF-1R) activates the PI3K/AKT and RAS-RAF-MAPK signaling pathways, and plays a critical role in proliferation, survival, vascularization, and invasion of tumor cells [19,20]. This signaling is active in several CNS tumors including medulloblastoma and glioblastoma [21,22] and components of the pathway are amplified in up to 20% of DIPG [13]. Inhibiting the IGF-1R pathway has been shown to reduce tumor progression, and halt metastasis formation in animal models of Ewing's sarcoma and glioblastoma [23-25].

BMS-754807 is a potent and reversible small molecule tyrosine kinase inhibitor of the IGF-1R. Targeting IGF-1R/IR signaling with BMS-754807 has resulted in cell growth inhibition in several pediatric tumor types, in vitro and in vivo [26,27]. Here we report the anti-tumor activity of BMS-754807 in a genetically engineered DIPG preclinical mouse model. In vitro BMS-754807 was found to have significant cytotoxic effects at clinically achievable concentrations with an IC₅₀ of 1.9 μM, and significant inhibition of proliferation at concentrations of 0.1 μM. In vivo we show that systemic administration of BMS-754807 to DIPG-bearing mice induces significant apoptosis in the tumor. Together, these results demonstrate that IGF-1R inhibition by BMS 754807 may provide much needed clinical benefit for children with DIPG and should be prioritized for translation into a future clinical trial.

### Materials and Methods

### DIPG Tumor Development:

Murine DIPGs were generated using a previously published genetic mouse model, which uses the RCAS/tv-a system to overexpress PDGF-B and delete p53 in brainstem nestin progenitors [28,29]. DF1 cells were purchased from ATCC and cultured in DMEM (ATCC) supplemented with 10% FBS, 2 mM L-glutamine, 100 units/mL penicillin and 100 μg/mL streptomycin, and incubated at 39° C. and 5% CO₂. Cells were transfected with RCAS plasmids (RCAS-PDGF-B, RCAS-Cre) using Fugene 6 or X-TremeGENE 9 (Roche) per the manufacturer's instructions. Cells were used for injections after being passaged at least 6 times from the time of transfection. 1 μL (10⁵ cells) of DF1 cells (ATCC Manassas, Va.) expressing RCAS-PDGF-B and RCAS-Cre were injected intracranially into nestin tv-a (Ntv-a) p53^{fl/fl} mice within 72 h of birth to generate brainstem gliomas. Mice were euthanized with CO₂ upon appearance of symptoms of brain tumor development (weight loss, lethargy, head tilt, or hydrocephalus) in accordance with Duke University IACUC protocol. Brain tissue from these animals was extracted, and either fixed in 10% neutral buffered formalin and paraffin embedded, or used to generate tumor cell lines.

### Neurosphere Cell Culture Development:

To generate DIPG neurospheres, extracted whole tumors were incubated with 5 mL papain solution (Papain 4.6 mg; EDTA 0.9 mg; Cysteine 0.9 mg; 5 mL Earls Balanced Salt Solution) plus 304 DNase (Sigma Aldrich). The solution was then triturated 30 times and incubated for 15 minutes at 37° C. The solution was again triturated 30 times and 2 ml of Ovomucoid solution was added (Ovomucoid stock solution: Ovomucoid protein 10 mg; 20 μL DNase); then incubated at 37° C. and diluted in 14 mL of Neurocult (basal) media (Stem Cell #05700). The solution was then centrifuged for 5 minutes at 1100 rpm at room temperature. The supernatant was then removed. 5004 of Ovomucoid solution and 5 ml of Neurocult (basal) media was added to the pellet, triturated 30 times and centrifuged an additional 5 minutes at 600 rpm. Resultant supernatant was collected and pellet washed (the addition of the Ovomucoid solution through the pellet washing steps were repeated in triplicate). Aggregate supernatant was filtered and plated at density of 500,000 cells per 25 cm² flasks in 6 ml of neurosphere media (per 50 ml total volume: 44.5 mL Neurocult (basal) media Stem Cell #05700; 10% proliferation supplement Stem Cell #05701; Pen-strep 1% Invitrogen #15140-122; Human basic FGF, 20 ng/mL Invitrogen #13256-029; Human EGF, 10 ng/mL Invitrogen #PHG0314; Heparin, 2 μg/mL Stem Cell #07980). Cells were incubated at 37° C. and split as required approximately once a week.

### High Throughput Drug Screen:

Primary cultures of DIPG neurospheres were added to 384 well plates pre-plated with 60 known drugs in an increasing gradient of concentrations. Drugs included in the screen were selected based on known DIPG target overexpression, and current availability for use in pediatric clinical testing. After 48 hours of incubation, CellTiter-Glo cell viability assays were completed, and the IC₅₀ for each drug was determined based on luminescence.

### Target Inhibition Map (TIM) Modeling:

The TIM method is a predictive modeling approach for tumor drug sensitivity [30,31]. As previously described [30,31] this model selects optimal drug targets for individualized tumors based on IC₅₀ data generated from a high throughput drug screen. This approach generates the TIM circuit, which is used as a visual representation of effective treatments inferred from the functional data. The resulting circuit is read as follows: by inhibiting a target in the TIM circuit, the line directly beneath its name is broken; if there exists no path from X (the origin) to Y (tumor proliferation), the target inhibition combination will be effective. This results in OR relationships between blocks (where if one block is inhibited, the treatment is effective) and relationships within blocks where all targets need to be inhibited for the block to be effective.

### Cellular Viability and Proliferation Assays:

DIPG neurospheres were placed in 96 well plates in neurosphere media at a density of 50,000 cells/1504 media and incubated for 48 hours with BMS-754807 (Active Biochemicals), OSI-906 (Selleck Chemicals), or matched DMSO control. A bromodeoxyuridine (BrdU) based cell proliferation ELISA assay kit (Roche) was used for proliferation and processed according to the manufacturer's protocol. Absorbance was read using Molecular Devices Versa Max Tunable Microplate reader at 370 and 492 nM. Viability was measured with CellTiter-Glo Luminescent Cell Viability Assay (# G7572, Promega) per manufacturer's protocol, luminescence values were obtained using Turner Biosystems Modulus Microplate luminometer (model 9300-062). All values are given as mean+/−SD of at least 3 independent experiments.

### Western Blot Analysis:

Cells were cultured for 1 or 4 hours with BMS-754807 or OSI-906 at 0.1 μM or 1 μM concentrations, with matched controls. IGF-1R ligand was added at 10 nmol/L concentration for 15 minutes prior to cell collection. Cells were lysed in nuclear lysis buffer containing: 50 mM Tris pH 7.5, 0.5M NaCl, 1% NP-40, 1% DOC, 0.1% SDS, 2 mM EDTA, protease inhibitor cocktail and phosphatase inhibitor cocktail 2 (Sigma). Protein concentrations of total cell lysates or total tissue lysates were determined using BCA assay kit (Pierce). Lysates were resolved by SDS-PAGE (Invitrogen) and transferred to nitrocellulose membranes (Bio-Rad Laboratories). After blocking in Odyssey Blocking Buffer with 0.1% Tween 20 (Li-Cor Biosciences), membranes were incubated with antibodies for the following targets of interest: phospho-IGF-1R 1:1000 (#3024), phospho-Akt^{ser473} 1:2000 (#4060), IGF-1R 1:1000 (#3018), Akt 1:1000 (#4685), and actin 1:500 (# sc-1616). All antibodies were purchased from Cell Signaling except Actin, which was purchased from Santa Cruz Biotech. Membranes were incubated with the appropriate labeled secondary antibodies, and protein was visualized and quantified using Li-Cor Biosciences Odyssey Infrared Imaging system (Li-Cor Biosciences). Inhibition was calculated by comparing the ratio of phosphorylation signals in untreated versus treated samples after normalization to actin. All values are given as mean+/−SD of at least 3 independent experiments.

### In Vivo Anti-Tumor Activity:

All experiments were performed under the Institutional Animal Care and Use Committee and Institutional Review Board of Duke University approved protocol (A239-10-9 and A214-13-8). Mice were maintained under barrier conditions. Nestin tv-a (Ntv-a) p53^{fl/fl} mice were injected with DF1 cells expressing RCAS-PDGF-B+RCAS-Cre as described above. Three weeks post injection; mice were randomly assigned to receive equal volume intraperitoneal injection of BMS-754807 or vehicle control. BMS-754807 was dissolved 2.5 mg/ml in a mixture of polyethylene glycol 400 (PEG₄₀₀ 4:1 H₂O). Mice were treated with 25 mg/kg BID for 6 doses. Loss of 25% of body weight, lethargy, head tilt, or seizure development was used as euthanasia criteria during the treatment window. Four hours after completion of the 6^{th} dose, treated animals were euthanized and tumor tissue was extracted as described above.

### Immunohistochemistry:

Tumor samples embedded in paraffin were then sectioned on a Leica RM2235 microtome at Sum thickness. Immunohistochemistry was performed using Discovery XT (Ventana Medical Systems). Sections were immunostained for cleaved caspase 3 (Asp175) (Cell Signaling #9661) and anti-phospho-histone H3 (Ser10) (Millipore 06-570) to allow for evaluation of apoptosis and proliferation, respectively.

### Digital Image Acquisition:

Digital images of microscopic fields of brain tissue were acquired with a Leica DMLB microscope, Leica digital camera and Leica Application Suite Version 3.7 (Leica; Buffalo Grove, Ill.) at 40× magnification. Camera settings were as follows: 381.0 ms exposure time, gain of 1.5×, saturation of 1.50, and gamma of 0.87. White balancing was completed before image acquisition. Coupled with the eye piece magnification of 10×, the final magnification of the images was 400×, with a resolution of 2592×1944 pixels and 63.1 μm² per pixel.

### Image Analysis:

Image analysis was completed using MetaMorph 6.1 software (Universal Imaging Corp.; Downingtown, Pa.) to quantify immunostaining on previously imaged sections for each injected animal. These images were thresholded using the HSI color representation method in the Set Color Threshold window. This method has previously been validated by Maximova et. al. [32]. This is a multidimensional evaluation of the hue, saturation, and intensity of each individual pixel making up an image with a range from 0-255. Once color thresholds had been established that were able to define all nucleated cells and only positively labeled cells, each of the ten images per injected animal were quantified for (per 40× high powered field): total nucleated cell area, total nucleated cell count, total positive cell area, total positive cell count. These data were exported to a Microsoft Excel spreadsheet where the following analyses were performed on each image: percent area of positive labeling, positive cell count labeling, and percent positive cell count number as a percentage of total cell number. The averages for each animal of each of these parameters were then transferred to GraphPad Prism 5 software (GraphPad Software, San Diego, Calif.) where a Mann-Whitney-U t-test was performed on each analyzed parameter to determine statistical significance.

### Results: High Throughput In Vitro Drug Screen Identifies IGF-1R as a Target of Interest for DIPG.

Secondary to the limited number of patients with DIPGs with available biopsy or autopsy generated tissue samples, there exists a significant backlog of novel therapeutics to potentially test in clinical trials. Therefore a large preclinical in vitro high throughput screening of currently available therapeutics was needed to elucidate small molecule inhibitors which may provide the greatest clinical yield. To complete this drug screen, primary cultures of nestin tv-a (Ntv-a) p53 deficient DIPG neurospheres were added to plates with 60 known drug samples in an increasing gradient of concentrations. Those included were selected based on known DIPG target overexpression, as well as drugs that are currently in other Children's Oncology Group trials or have future potential to enter clinical testing. From the initial 60 drugs tested, 8 cytocidal drugs were identified with cytocidal defined as <15% cell viability at 10 μM. The top hits in the drug screen in order of increasing IC₅₀ were the following: BMS-754807, Obatoclax, SNS-032, Carfilzomib, OSI-906, BIX-01294, Crizontinib, Sunitinib. Specific targets of the positive hits from the drug screen are listed, in order of ascending IC₅₀ (Table 1).

TABLE 1
Drug Screening Top Hits

| | In Vitro Absolute | |
|---|---|---|
| Drug | IC50 (uM) | Target |
| Obatoclax | 0.159063117 | Bcl |
| Crizontinib | 6.226315716 | ALK |
| SNS-032 | 0.505366589 | CDK |
| Carfilzomib | 0.833592656 | Proteasome |
| BIX-01294 | 3.954894512 | Histone-lysine Methyltranferase |
| BMS-754807 | 0.097410691 | IGF1-R |
| Sunitinib | 6.924579436 | VEGF-R, PDGF-R, Kit |
| OSI-906 | 1.738317472 | IGF1-R |

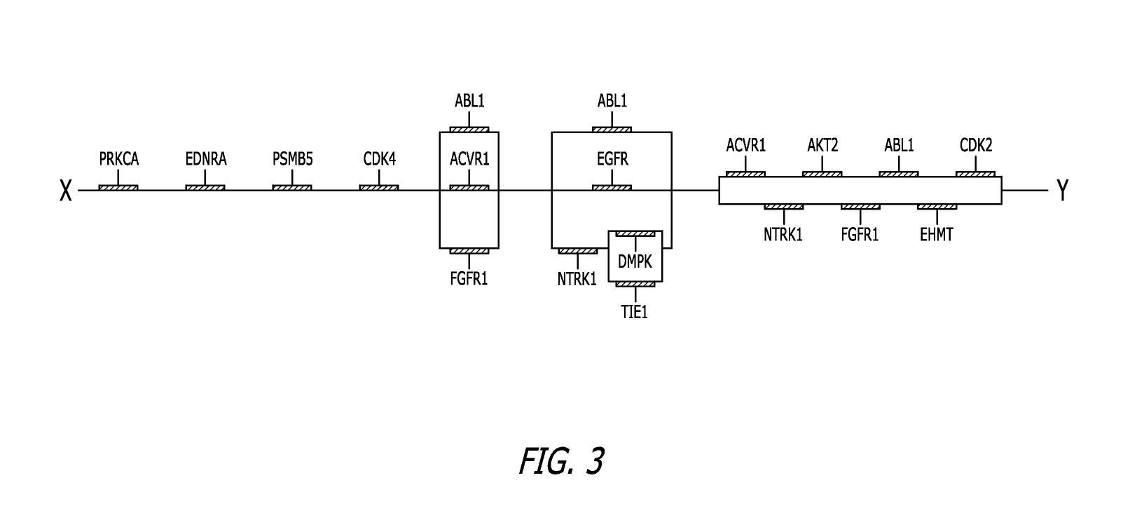

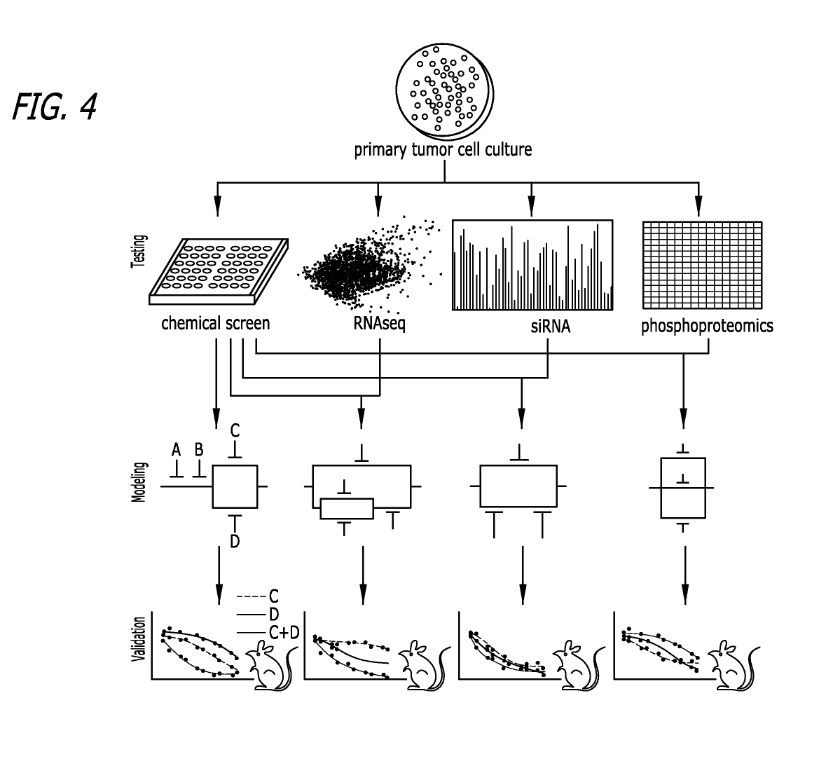

Subsequently, a probabilistic target inhibition map (PTIM) was generated, which highlighted IGF-IR as a target of interest (FIG. 3). This hit is due primarily to the effectiveness of BMS-754807 (an IGF-IR inhibitor, but one which is much more promiscuous) and OSI-906 (a significantly more selective IGF-1R inhibitor), which have low IC₅₀ values. This leads to IGF-1R being a single point of failure, as indicated by IGF-1R being in an independent block. Targeting IGF-1R either independently or in combination with other targets may be an effective treatment for DIPG. Other single points of failure include PSMB5, a proteasome inhibitor, and several HDAC targets. However, in clinical usage, it is likely that two of more of the HDAC targets would need to be inhibited to guarantee an effective treatment. Similarly, the CDK targets (2, 7, and 9) are highly effective as a block combination, but some CDK targets (in particular CDK2) may be highly effective independently. However, tested CDK 2 inhibitor SNS-032 is no longer in clinical development, therefore BMS-754807 was further validated in both in vitro and in vivo studies.

### In Vitro, BMS-754807 Reduces Proliferation and Viability of DIPG Neurospheres in a Dose Dependent Manner.

Results of the high-throughput drug screen are first confirmed with repeated proliferation and viability assays using BMS-754807. CellTiter-Glo assays showed significant reduction in DIPG neurospheres at drug concentrations of 0.1 μM and higher, with IC₅₀ of 0.19 μM. BMS-754807 also significantly inhibited proliferation as demonstrated by our BrdU assay with an IC₅₀ of 1.8 μM.

As the DIPG mouse model is driven by PDGF-B, it is unexpected that two of the top hits in the drug screen were inhibitors of IGF-1R signaling and not inhibitors of PDGFR signaling (AP24534 (ponatinib), dasatinib, panzopanib, sorafenib). Therefore, western blot is performed on whole tumor lysates from our mouse model to confirm that IGF-1R signaling is present in vivo. Different levels of IGF-1R activation across tumor samples was observed.

Next it was determined if BMS 754807 inhibits IGF-1R and one of its downstream targets, AKT in a dose dependent manner in our DIPG derived neurospheres. In the absence of IGF-1 ligand stimulation, the DIPG neurospheres demonstrated low to undetectable pIGF-1R levels likely due to the absence of IGF ligands in the serum-free media (data not shown). Therefore western blot analysis was completed with IGF-1 stimulation at 10 nmol/L concentration. Inhibition of both pAKT and pIGF-1R was observed after 4 hours of drug exposure with BMS 754807 at concentrations of 0.1 μm and higher.

Although BMS 754807 inhibits IGF1-R activation, it is a relatively non-specific drug and the reduction in AKT phophorylation as well as the cytotoxic effects seen in the screen may potentially be due to inhibition of one of its other important targets listed here: insulin receptor, MET, ALK, TRKA, TRKB, AURKA, AURKB, JAK2, CDK2 (Table 2).

TABLE 2
Target Selectivity of BMS-754807

| Target | K_{d} (nM) BMS-754807 |
|---|---|
| IGF1-R | 1.8 |
| Insulin R | 1.7 |
| MET | 5.6 |
| ALK | 5.7 |
| TRKA | 7.4 |
| TRKB | 4.1 |
| AURKA | 9 |
| AURKB | 25 |
| JAK2 | 347 |
| CDK2 | 795 |

In order to more specifically demonstrate that IGF-1R mediated pathway inhibition is the key mechanism of action for the observed reduction in viability and proliferation, a more specific IGF-1R targeted drug, OSI-906, was also evaluated in our DIPG neurosphere cultures. DIPG neurosphere proliferation and viability were significantly inhibited with OSI-906 concentrations of 1 μM and above, with an IC₅₀ of 2.5 and 2.4 μM respectively. Western blot target evaluation showed a 73% reduction in pIGF-1R and 69% reduction in pAKT expression following 4 hours of drug exposure at 1 μM and higher. In summary, both BMS 754807 and OSI 906 inhibit IGF-1R and AKT activation in a dose dependent manner in our DIPG model at doses that also demonstrate cytotoxicity. As drug delivery into the tumor in DIPG is an important variable, next we evaluated BMS 754807 in vivo.

### BMS-754807 Significantly Increases Apoptosis In Vivo.

DIPG-bearing mice were treated with a short course of BMS 754807 starting at 3 weeks post tumor induction at a time point where tumors have already formed (based on prior studies) but before the mice develop symptoms. DIPG tumors were collected from mice treated with either BMS-754807 or matched vehicle controls (n=10 per group) and immunostained for phosphorylated histone-3 at serine 10 (a marker for tumor proliferation) and cleaved caspase 3 (a marker of apoptosis). It was observed that BMS-754807 did not significantly reduce DIPG proliferation (11.54+/−2.58 mean vehicle versus 9.632+/−2.827 mean BMS754807 treated p=0.8421 in percentage of cell per high powered filed) but did significantly induce apoptosis in vivo (06.571+/−0.1561 mean vehicle versus 1.063+/−0.1925 mean BMS754807 treated p=0.0435 in percentage of cell per high powered filed).

As the result of a 60 drug in vitro high-throughput screen, IGF-1R was identified as a therapeutic target in the exemplary DIPG model of the present invention. IGF-1R is a transmembrane tyrosine kinase growth factor receptor. When activated, it regulates cell proliferation and cell survival. BMS-754807, an IGF-1R inhibitor, was evaluated in this study to determine potential efficacy in treating children with DIPGs. While BMS-754807 is currently undergoing clinical trials for the treatment of breast, head and neck, and other solid tumors, there are no active trials open for enrollment for children with these lethal brain tumors (Clinicaltrials.gov). Therefore, the rationale for this study was to use our genetically engineered mouse modeling system to determine the efficacy of BMS-754807 in pediatric DIPG.

It is established that our PDGF-driven DIPG mouse model has activated IGF-1R signaling. Given the relatively low in vitro IC₅₀ of BMS-754807 of 0.09 μM in the high throughput drug screen, we set out to investigate whether BMS 754807 should be prioritized for clinical trials for children with DIPG. Our observations suggest that p53 deficient DIPG cells require IGF-1R mediated signaling for survival and growth in vitro. OSI-906, a more selective IGF-1R inhibitor was used to strengthen the evidence that IGF-1R is a therapeutic target in our model as BMS 754807 inhibits other targets such as MET, ALK, TRKA, TRKB, AURKB, JAK2, IR, and CDK2. The results of the OSI-906 experiments suggest that at least some of the anti-tumor effect of BMS-758407 is likely through inhibition of IGF-1R signaling.

The in vitro experiments were conducted in serum-free conditions with several independent murine DIPGs induced by PDGF-B and p53 loss. The serum-free culture conditions selects for stem-like tumor cells given the addition of EGF and FGF to the media and the lack of serum [33]. The increased apoptosis observed in vivo in DIPG-bearing mice treated with BMS-754807 supports observations in vitro. These data support the notion that serum-free culture conditions are appropriate to test novel therapeutics in DIPG. Additionally, the results also suggest that BMS-754807 was able to cross the blood brain barrier as in vivo treatment with BMS-754807 in tumor bearing mice resulted in a significant increase in apoptosis after a short-treatment course. Furthermore, BMS-754807 was well tolerated by mice undergoing treatment.

Overall, as the exemplary DIPG model closely mirrors the human condition, this study suggests that BMS-754807 should be evaluated in clinical trials for children with IGF-1R overexpressing DIPGs. The results presented here also predicate the need for biopsy guided therapeutic intervention based on an individual child's tumor genetic make-up. By evaluating DIPG biopsy tissue for IGF-1R expression levels, those children that are potential benefactors of BMS-754807 treatment can be identified and effectively treated. Future experimental direction would include determining if the short-term treatment efficacy with BMS-754807 observed in this study would prolong survival in PDGF-B driven, p53 deficient DIPGs. In addition, recent studies have shown increased efficacy with combined PDGFR α/β and IGF1-R targeted therapy in pediatric GBM cell lines, and may be a potential therapeutic combination for future studies [34].

In conclusion, in vitro and in vivo drug evaluations in a genetic mouse model of DIPG can help prioritize novel drug therapies. These findings demonstrate that BMS-754807, a potent IGF-1R inhibitor, can significantly reduce both viability and proliferation of DIPG tumor cells at doses of 0.1 μM and higher, confirmed with preliminary in vivo studies. These results are promising and imply that IGF-1R is a potential therapeutic target meriting further study. Future experiments aim to follow-up with a survival study and identify other therapies that may synergize with BMS 754807.

SUPPLEMENTAL TABLE 1

| Drug Tested: | Target (s): |
|---|---|
| Lapatinib | Egfr, ErbB2 |
| 5-Aza-2 deoxycytidine | DNA-methyl transferase |
| (Decitabine) | |
| Bortezomib | Proteasome |
| Cilengitide | αvβ3 and αvβ5 integrins |
| Dasatinib | C-Kit, PDGFR, SFK, Bcr-Abl |
| MK2206 | Akt1, 2, 3 |
| MLN8237 | Aurora kinase A |
| Pazopanib | VEGFRs, c-KIT, PDGFR beta |
| R115777 (tipifarnib, | Enzyme(Farnesyl transferase - Enzyme) |
| Zarnestra) | |
| RO4929097 | Notch |
| SAHA (Vorinostat) | HDAC |
| Sorafenib | VEGFR, PDGFR, RAF |
| Temsirolimus (sirolimus | mTOR |
| prodrug & active itself) | |
| Crizotinib | C-Met, ALK |
| AUY922 | Hsp90 |
| INCB018424(Ruxolitinib) | Jak1, Jak2 |
| OSI-906 | IGF1R |
| ABT-888 | PARP-1 and PARP-2 |
| AZD6244 (Selumetinib) | MEK1/2 inhibitor |
| Cediranib | VEGFR |
| GDC-0449 | Smoothened (Hedgenog pathway) |
| Entinostat | HDAC 1, 2 |
| Obatoclax mesylate | BCL2, BH3 |
| (GX15-070MS) | |
| SCH-727965 | CDK2, CDK5, CDK1, and CDK9 |
| CC-5013 (lenalidomide, | Angiogenesis(VEGF - Unknown), |
| Revlimid) | Immune System(T-cell, IL-2, IL-6, |
| | IL-10, IL-12, IL-1beta, TNF-alpha, |
| | IFN-gamma - Unknown) |
| Fenretinide | retinoid receptor |
| Flavopiridol (alvocidib) | Cell Cycle(Cyclin-dependent kinases - |
| | Enzyme), Cell Cycle(Cyclin-dependent |
| | kinases - Protein) |
| Fostamatinib (R788) | SYK kinase |
| Saracatinib (AZD0530) | Src Family Kinases |
| N-acetyl cysteine | Reactive oxygen species |
| Enzastaurin | Protein Kinase Cβ |
| Panobinostat | HDAC |
| BMS-387032 | CDK2, CDK7, CDK9 |
| EKB-569 | Egfr, ErbB2 |
| VX-680 | Aurora kinases |
| Sodium butyrate | HDAC |
| 4-hydroxy-tamoxifen | estrogen receptor |
| AP24534 (Ponatinib) | BCR-ABL, VEGFR2, FGFR1, PDGFRα |
| ATRA | differentiating agent |
| AZD1152-HQPA (Barasertib) | Aurora kinase B |
| BMS-754807 | IGF1R |
| Carfilzomib | Proteasome |
| Imelelstat (GRN 163L) | Telomerase inhibitor |
| PF-04554878 | FAK inhibitor |
| Quinacrine | autophagy pathway |
| RO5045337 | MDM2 |
| Trichostatin A | HDAC |
| Vandetanib | VEGFR-2, EGFR, RET |
| ZD4054 | Endothelin A receptor |
| AP26113 | ALK, EGFR |
| BEZ 235 | PI3K, mTOR |
| BIX-01294 | Histone-lysine methyltransferase |
| GANT61 | Hedgehog (Gli1) |
| Nilotinib | PDGF-R, c-Kit, BCR-ABL |
| SB-431542 | TGF beta |
| SJ-172550 | MDMX |
| SP600125 | JNK 1, 2 and 3 |
| vemurafenib | BRAF |
| VER-155008 | Hsp90, Hsp70 |

### Example 2

### Probablistic Boolean Modeling of Target Synergies from Integrated Chemical Screens and Genomics

Using an unbiased GSK orphan kinome screening drug library, as well as additional biological data generated from a single test patient sample, nonlinear probabilistic Boolean models describe the evidence-based mechanisms likely resulting in growth inhibition following drug application. The generated models were independent of existing research on disease of choice, alveolar rhabdomyosarcoma, and were generated using only data created exclusively for the pseudopatient of this study. Based primarily on the screen data, and refined by incorporation of transcription abundance data, two nonlinear models were selected for in vitro validation. Both models identified targets implicated in the IGF1R signaling pathway, consistent with existing research on alveolar rhabdomyosarcoma. The expression-naïve PTIM model identified a combination of IGF1R/INSR/PRKACA as being a group of synergistic mechanisms. By informing the model with transcript data, a combination of IGF1R/PI3KCA was identified as an effective combination of mechanisms. In the course of biological validation, multiple drug screens were the basis for the testing of U23674. Differences between the chemical screen results, mostly due to the different nature of the targets and interactions, point to a crucial decision to make concerning chemical selections for drug screens. Chemical selection can potentially point towards different mechanisms.

These mechanisms were tested in vitro to show the presence of synergistic effects; both models generated highly synergistic drug combinations. The combinations were retested using significantly lower effective dosages to support the synergistic behavior resulting from the target mechanisms used to select the drugs. Due to the significantly higher level of growth inhibition associated with the IGF1R/PI3KCA model in vitro, it was selected as the basis for a four-arm orthotropic allograft experiment. The experimental results indicate a significant shift in survival for the combination mice as compared to any of the other groups, supporting the potential for the PTIM model to predict target synergy and guide combination therapy design in vivo. In addition, the similarity of survival times for the single agent and control groups support the hypothesis that the PTIM model can identify combination synergy of individually ineffective targets, promoting the use of a model which can identify multi-target drivers of cancer. Based on these results, the potential exists for PTIM-based modeling to be developed further as a clinically-usable approach to combination therapy design.

### Results of GSK Orphan Kinome Screen.

For the present example, a low passage primary tumor cell culture of a single murine alveolar rhabdomyosarcoma, designated U23674, was as a robust experimental pseudo-patient. A total of 305 compounds were screened against U23674. Of these 305 compounds, 40 (13%) showed sensitivity as defined by causing at least 50% growth inhibition in vitro at the highest concentration tested. As these are experimental compounds, the lower rate of sensitive drugs is expected. Most targets, when considered as independent mechanistic agents, had low occurrence in sensitive compounds. Targets in the GSK screen had occurrence rate in sensitive compounds of μ=10.56%, σ=9.73%. A total of 13 of 208 kinase targets have occurrence at a rate greater than μ+2σ. The average coverage of drug targets was 24.03 compounds/target.

### Results of Roche Orphan Kinome Screen.

A total of 223 compounds were screened against U23674. Of these 223 compounds, 22 (9.87%) showed sensitivity as defined by causing at least 50% growth inhibition in vitro at the highest concentration tested. As with the GSK Orphan Kinome screen, the experimental nature of the tool compounds informs the lower rate of sensitive drugs. As the two screens targeted different targets in different combinations, there will naturally be variation in successful targets and drug sensitivity rates. Less information is available concerning the kinase targets of several of the drugs on this screen. However, 103 kinase targets were found to be associated with the 22 drugs which had sensitivities

### Results of Pediatric Preclinical Screen Version 2.1 (PPTI Screen) Primary Tumor Cell Culture.

A total of 60 compounds were screened against U23674. Of the 54 compounds with known sensitivity profiles, 27 (50%) displayed in vitro sensitivity at the highest concentration tested. Targets in the PPTI screen had an occurrence rate in sensitive compounds of μ=23.06%, σ=31.65%. A total of 72 of 425 kinase targets had an occurrence rate greater than μ+0.75σ. The average coverage of drug targets was 1.86 compounds/target. Considering only those targets with coverage twice the average, the occurrence rate in sensitive compounds was μ=30.49%, σ=19.49%, resulting in an average coverage of 5.2 compounds/target. A total of 18 of 80 highly covered kinase targets had an occurrence rate in sensitive compounds at a rate greater than μ+a. Table 2 provides the sensitivity values (as IC50 values) for the PPTI screen.

### Results of RNAseq Analysis.

RNA deep sequencing (RNAseq) identified 23,339 unique, expressed mRNA transcription factors. RNAseq identified 4854 significantly (p<0.05) differentially expressed genes compared to regenerating adolescent murine skeletal muscle. Regenerating skeletal muscle is reasoned as an a appropriate control because rhabdomyosarcoma is often felt to be phenotypically similar to activated satellite cells/myoblasts, and rhabdomyosarcoma has in experimental systems been shown to have a myoblast origin (19, 20). Of these, 2166 proteins (9.28%) were overexpressed in U23674 relative to the regenerating muscle control and 2688 proteins are under-expressed relative to the control. Gene Ontology analysis was performed for the proteins identified in the different cases. Differentially activated processes in the tumor samples are: cellular nitrogen compound metabolic process, biosynthetic process, anatomical structure development, signal transduction, transport, cell differentiation, cellular protein modification process, response to stress, cellular component assembly, cell death. Suppressed processes in the tumor sample are: biosynthetic process, transport, cellular nitrogen compound metabolic process, anatomical structure development, signal transduction, cellular protein modification process, response to stress, cell differentiation, small molecule metabolic process, catabolic process. Each list constitutes the ten most significant over and under expressed processes based on differential gene expression.

### Results of Phosphoproteomics Analysis.

Samples were analyzed for protein and phosphoprotein levels using an antibody array assay provided as a service (Kinexus, Vancouver, British Columbia, Canada). Targets were identified as significantly differentially phosphorylated between two samples if the absolute value of the Z-score ratio was greater than 1. Consequently a total of 28 gene targets were identified as differentially phosphorylated or expressed in U23674 relative to at least one other sample (control damaged muscle as well as primary cell cultures representing metastatic alveolar rhabdomyosarcoma from a different mouse (U48484) and embryonal rhabdomyosaroma (U57810)). Of these 28, 7 are identified as being significantly different between the two duplicate lysates of U23674 and are considered to be unreliable. Interestingly, among these 7 are two Cyclin-dependent kinases, CDK2 and CDK6, which are both targets of interest in rhabdomyosarcoma. Among the remaining 21 targets, 10 are differentially phosphorylated only in other rhabdomyosarcomas and may represent variational differences between the different individuals (individual mice). Among the remaining 11 targets, 6 are under phosphorylated and 5 are overphosphorylated compared to at least one control sample. Three of the under expressed targets (Akt1, Jun, Prkaca) and three of the over expressed targets (Map2k1, Mapkapk2, Relb) are present in the MAPK signaling pathway, implicating this pathway as potentially important in the biology of U23674.

Results of RAPID siRNA Screen.

The RAPID siRNA screen of receptor tyrosine kinases performed on U23674 identified three highsensitivity kinase targets: Ntrk2, Pdgfrb, and EphB4. In (21) the results from this screen were further explored, and an Ephb4-Pdgfrb axis was explored in vitro. Briefly, expression of these receptor tyrosine kinases (RTKs) at the protein level was validated by immunoblotting, and RAPID screen results were validated using pooled EphB4 and Pdgfrb siRNA compared to a control mouse myoblast cell line (21). The compound dasatinib was used to test the efficacy of Ephb4-Pdgfrb inhibition in vitro. FIG. 10 shows the results of the RAPID siRNA screen on U23674 done at the time of the GSK screen. Three targets were identified using the criteria of having overall cell viabilities less than two standard deviations below the mean following siRNA knockdown: Ephb4, Pdgfrb and Ntrk2. Ephb4 had been implicated in 33% (1 of 3 EphB4 related drugs tested) for the PPTI screen; 7.14% (2 of 28 drugs tested) for the GSK screen; Ephb4 was not inhibited in any sensitive chemical agents on the Roche screen. Pdgfrb had been implicated in 14.3% (1 of 7 drugs tested) for the PPTI screen; 10.47% (11 of 105 drugs tested) in the GSK screen; Pdgfrb was not inhibited by any effective chemical probe compounds on the Roche screen. Ntrk2 had been implicated in 50% (2 of 4) in the PPTI screen; 7.9% (3 of 38 drugs tested) in the GSK screen; Ntrk2 was not targeted by chemical agents on the Roche screen. The prior validation of dasatinib sensitivity on U23674 has been shown to be consistent with an the Ephb4-Pdgfrb interrelated molecular mechanism of tumor cell growth (21); however, the lack of efficacy of sunitinib (PPTI screen), which also inhibits Ephb4-Pdgfrb, resulted in the target combination not appearing in the TIM modeling approach for the PPTI screen (despite the validation of this relationship in (21)). The GSK screen contained compounds which simultaneously inhibited these two targets, but the low sensitivity (IC50 ˜5000 nM, 2 of 23 drugs tested) led to it not being a target combination of interest. No effective compound in the Roche screen inhibited any of these targets.

### PTIM-Based Model Generation for U23674.

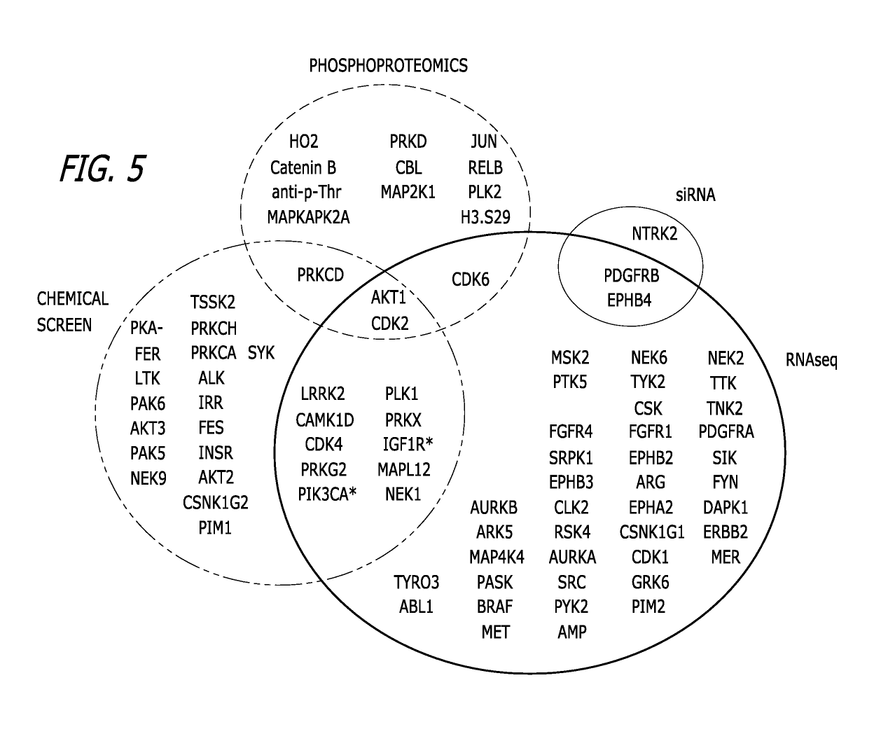

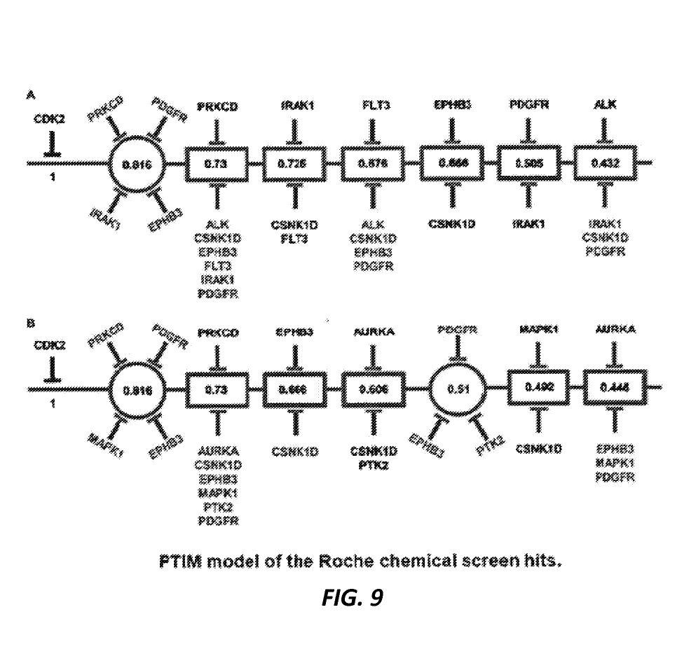

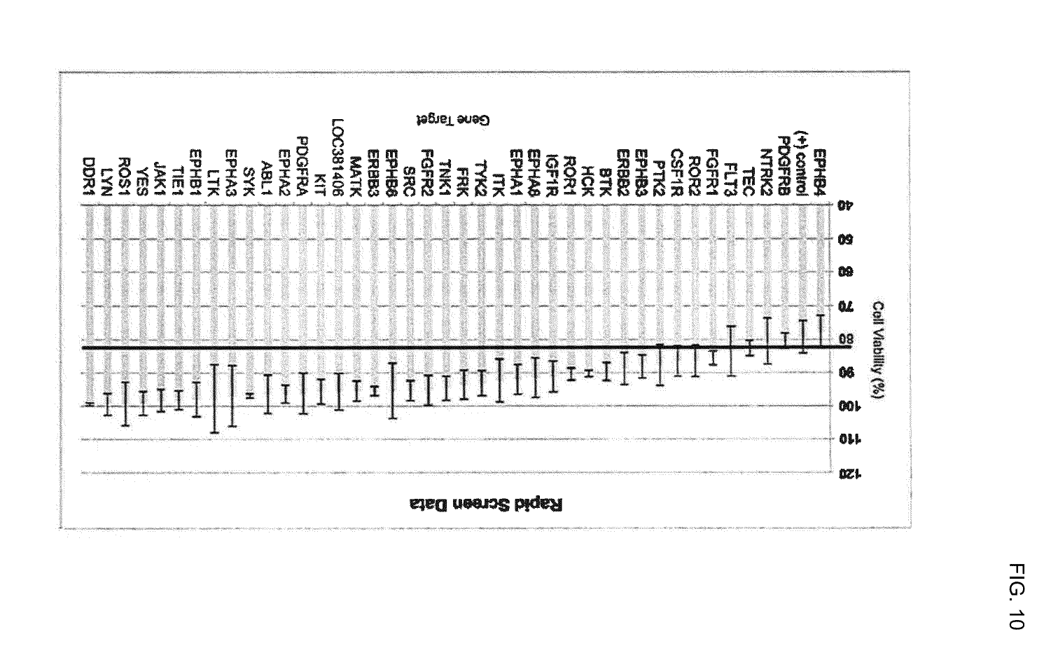

A total of 8 models were generated for the analysis and combination screening of U23674. Each of the two chemical screens were used as input data to the PTIM generation algorithm independently, and informed by the following datasets: RNAseq expression, RAPID siRNA screen, and Kinexus phosphoproteomics data. This results in a total of 4 models per chemical screen. FIG. 5 shows the targets identified by the various approaches and the intersecting targets of interest. The PPTI screen data was used to partially validate the accuracy of the compound sensitivities generated by the experimental GSK screen, and as such the GSK screen data was used to develop the combination screen validation experiments. Supplemental FIG. 9 details the targets identified by both drug screens, and commonalities therein. For selection of experiments to run, combinations of interest were identified from the resulting models. In particular, target combinations which could be satisfied by a combination of two kinase inhibitors were selected based on model recommendations. Tested combinations suggested by the PTIM method consists of two combinations of three targets. IGF1R+INSR+PRKACA is the target combination suggested by the raw GSK agents on the Roche screen. The prior validation of dasatinib sensitivity on U23674 has been shown to be consistent with an the Ephb4-Pdgfrb interrelated molecular mechanism of tumor cell growth (21); however, the lack of efficacy of sunitinib (PPTI screen), which also inhibits Ephb4-Pdgfrb, resulted in the target combination not appearing in the TIM modeling approach for the PPTI screen (despite the validation of this relationship in (21)). The GSK screen contained compounds which simultaneously inhibited these two targets, but the low sensitivity (IC50 ˜5000 nM, 2 of 23 drugs tested) led to it not being a target combination of interest. No effective compound in the Roche screen inhibited any of these targets.

### PTIM Modeling can Predict In Vivo Target Synergy and Guide Combination Therapy Design.

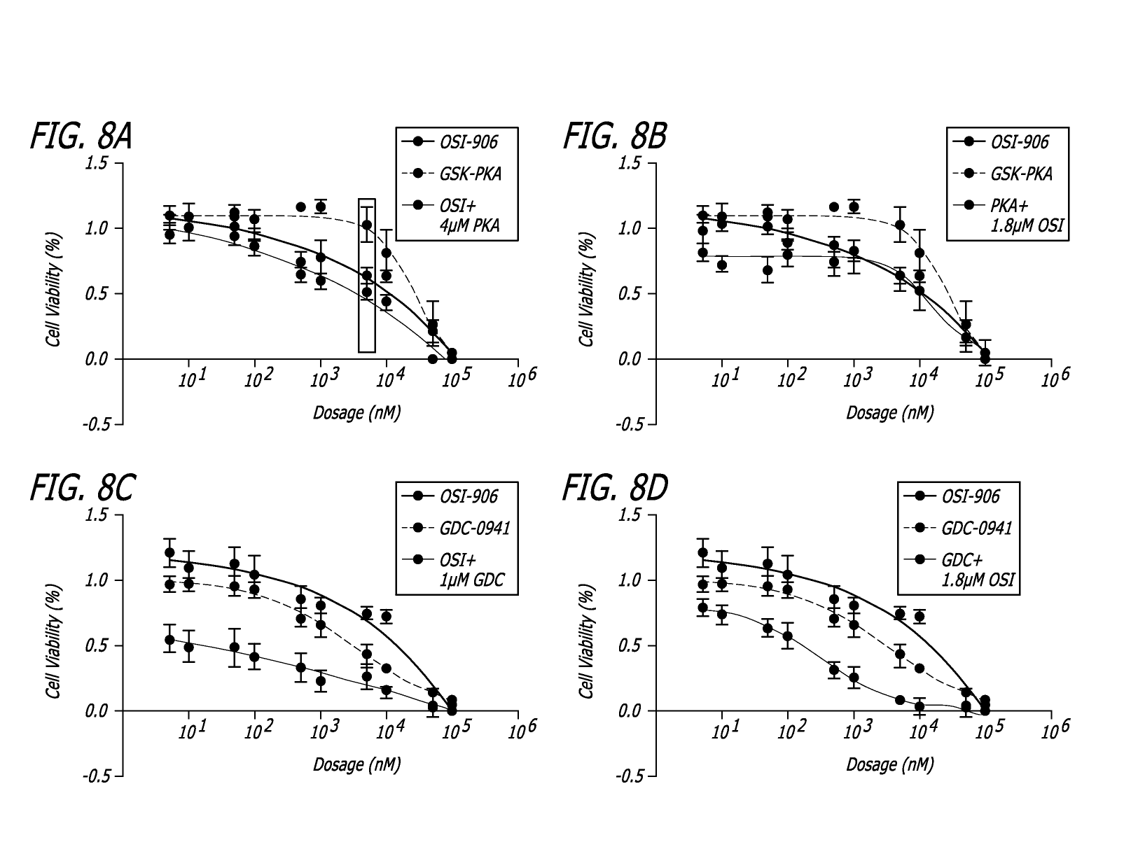

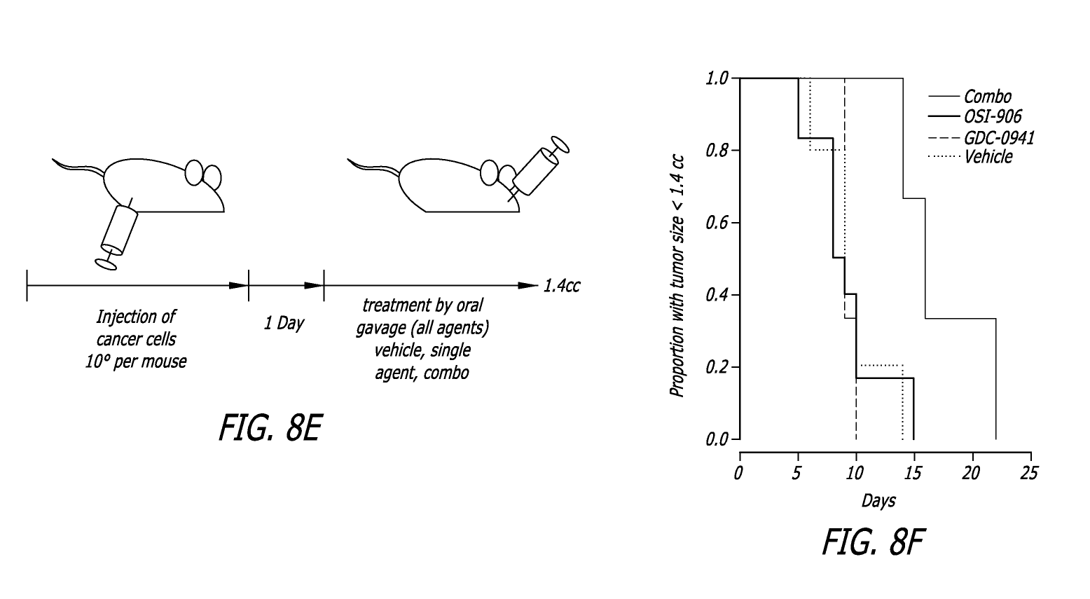

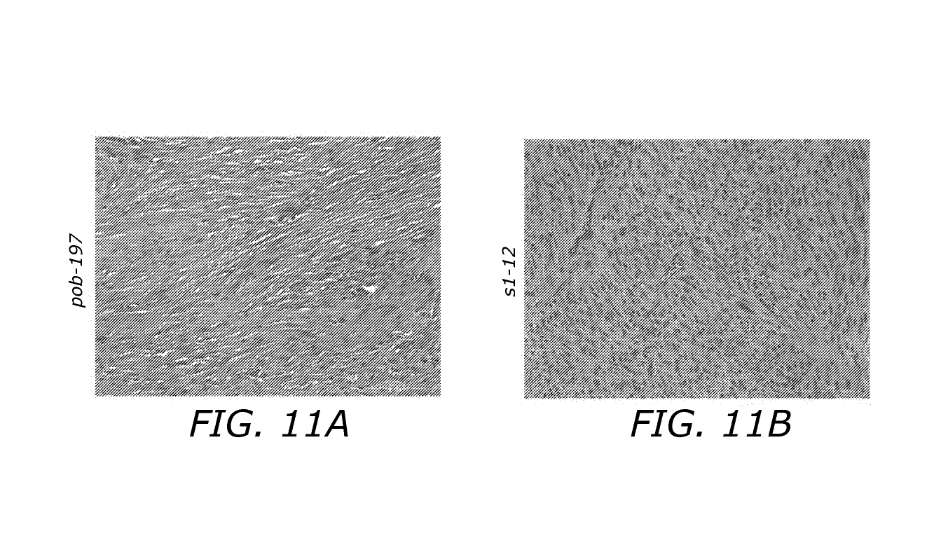

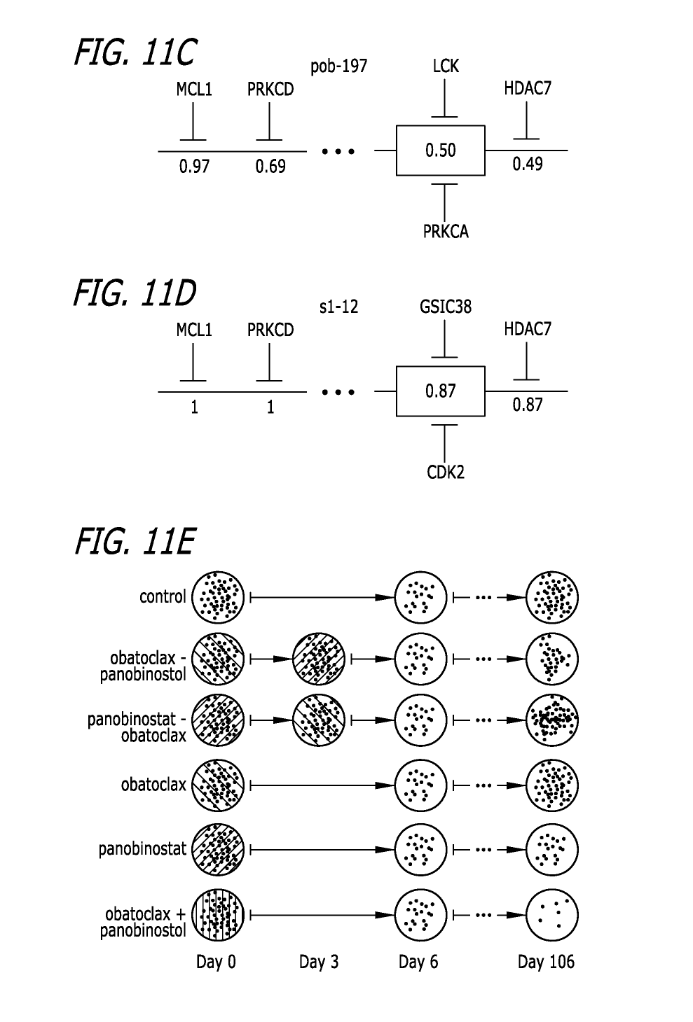

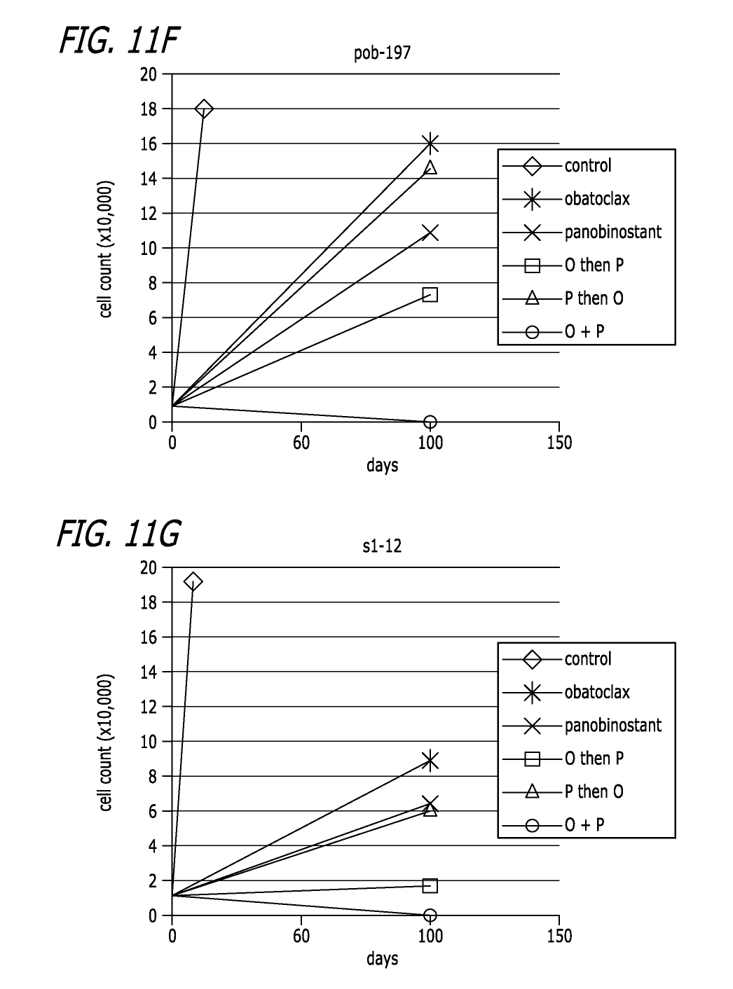

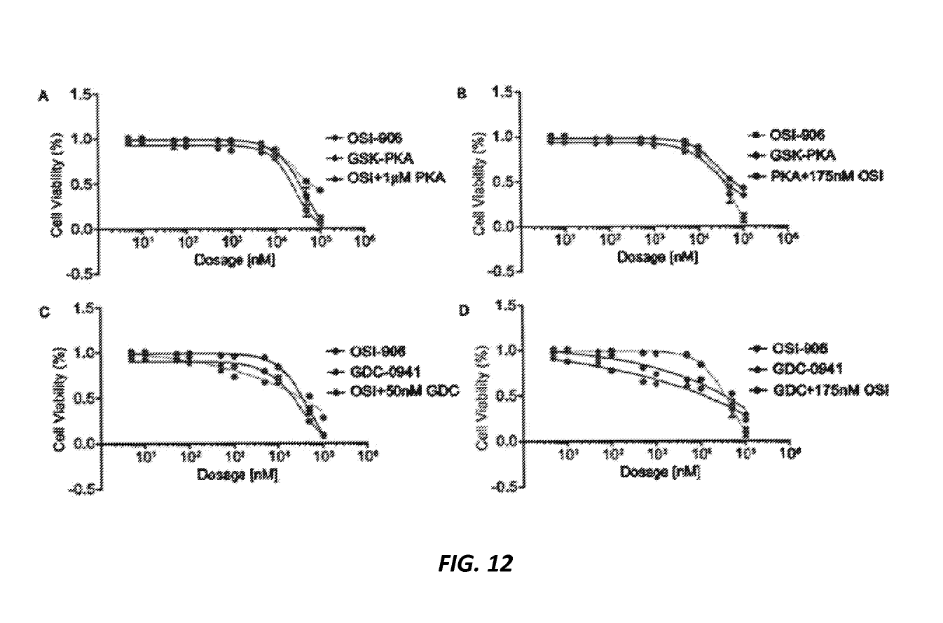

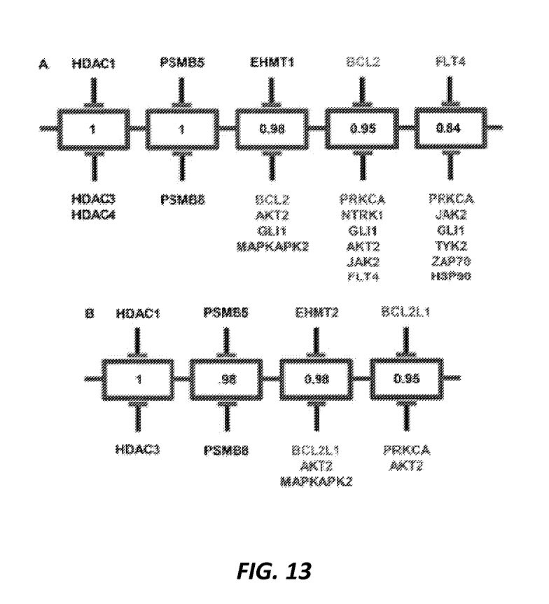

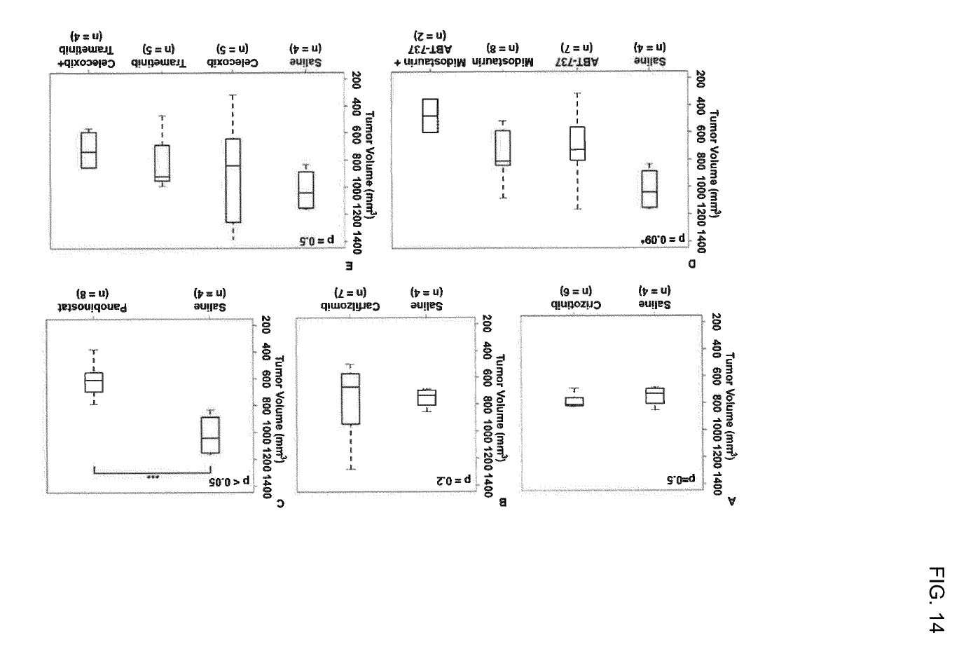

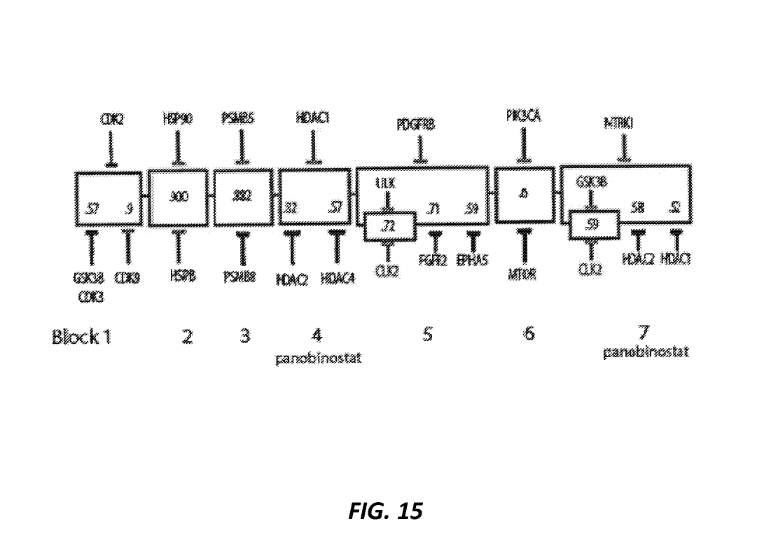

Following in vitro validation experiments, the combination from the RNAseq-informed PTIM was used to design a four-arm orthotropic allograft study. The selected combination of drugs was GDC-0941 (PIK3CA inhibitor) in combination with OSI-906 (IGF1R inhibitor). Kaplan-Meyer survival analysis for the four arms of the experiment are given in FIG. 8. Survival of mice administered combination therapy was extended compared to all other groups (p=0.079 after correction for multiple comparisons), whereas survival of mice treated with either single agent was no better than survival of mice only treated with vehicle (p>0.7 raw p-values for all compounds compared to vehicle, p>0.9 using the Tukey-Kramer method). The similarity between the single agent and control groups is of interest due to the predictive aims of the PTIM analysis method. A single block of the PTIM model represents two targets which individually will not lead to significant improvement in survival but which, when jointly inhibited, will provide a much more effective treatment. The similarity in survival between the two single agent groups and the control group, as well as the significant improvement in survival for the combination mice, supports the hypothesis that our computational model can identify synergistic combinations of targets where individual targets would have negligible efficacy. Similar value for predicting targets to prevent evolution of drug resistance by inhibiting targets in series is presented in FIG. 11 for related human and canine soft tissue sarcomas. Applicability of PTIM to human patient-derived xenografts is described further in FIGS. 13 and 14 and Supplemental Tables 2 and 3.

SUPPLEMENTAL TABLE 2

| | | log2 PCB209PDX |
|---|---|---|
| Gene | log2 PCB209 RPKM | RPKM |
| HDAC1 | 4.376054175 | 4.501063295 |
| HDAC3 | 4.157100317 | 3.760657415 |
| HDAC4 | 1.336111968 | 2.60566026 |
| PSMB5 | 7.110391434 | 6.893165599 |
| PSMB8:1 | 5.462490821 | 3.886852506 |
| EHMT1 | 2.87091703 | 3.213409499 |
| EHMT2:1 | 3.495963724 | 4.233581332 |
| BCL2 | 0.204391163 | 0.645517218 |
| BCL2L1 | 6.070619024 | 5.858585308 |
| AKT2 | 4.072294488 | 4.380154527 |
| GLI1 | 0.01564029 | 0.916553077 |
| PRKCA | 3.676820275 | 4.699979092 |
| NTRK1 | −1.017125039 | −1.138918058 |
| JAK2 | 2.446626783 | 2.72034414 |
| FLT4 | −2.656535324 | −9.587272661 |
| TYK2 | 3.822974825 | 4.324248966 |
| ZAP70 | 0.704960471 | −7.454822365 |
| HSP90AA1 | 7.498684887 | 8.735472083 |

### Methods.

The mouse primary tumor cell culture, U23674, was established from a tumor at its site of origin in a genetically engineered Myf6Cre,Pax3:Foxol,p53 mouse bearing alveolar rhabdomyosarcoma as previously described (19). The mouse primary tumor cell culture, U48484 was established from a pulmonary metastatic tumor of an biologically-independent Myf6Cre,Pax3:Foxol,p53 mouse bearing alveolar rhabdomyosarcoma as previously described (19). The mouse primary tumor cell culture, U57810 was established from the tumor site of origin of a Myf6Cre,p53 mouse bearing embryonal rhabdomyosarcoma. In brief, for each culture the tumor was broken into small pieces and digested with collagenase (10 mg/ml) overnight at 37° C. The dissociated cells were then incubated in Dulbecco's modified eagle's media (DMEM) supplemented with 10% fetal bovine serum (FBS) and 1% penicillin-streptomycin in 5% CO2 at 37° C. These experimental cell cultures were maintained in the same culture conditions as primary tumor cell cultures: DMEM supplemented with 10% (FBS) and 1% penicillin-streptomycin. The single-agent drug validation experiments and drug combination experiments were performed at passage 5.

### Chemical Screens.

Three chemical screens were applied to the U23674 primary tumor cell culture. The first screen was a custom 60 agent drug screen, denoted the Pediatric Preclinical Testing Initiative Screen Version 2.1 (PPTI screen), consisting of well-characterized target inhibitor compounds selected to provide a greater breadth of target combinations. Fifty-four of the 60 drugs on the chemical screen have a complete drug-target inhibition profile. The target inhibition profile is a quantified description of the inhibitory properties of the compound on the numerous protein targets preset on the PPTI screen. The second chemical screen is a GlaxoSmithKline open access Orphan Kinome-focused chemical screen consisting of 402 novel and newly characterized compounds (23, 24). These compounds have had target inhibition profiles quantified by Nanosyn Screening and Profiling Services. The assay provided inhibition properties of the 402 compounds on 300 protein targets. The compounds were tested at 100 nM and 1004 concentrations to bracket the drug-protein EC50 values. The final EC50 values used for analysis of the chemical screen results were inferred from the available data using a hill curve to predict the 50% inhibition point.

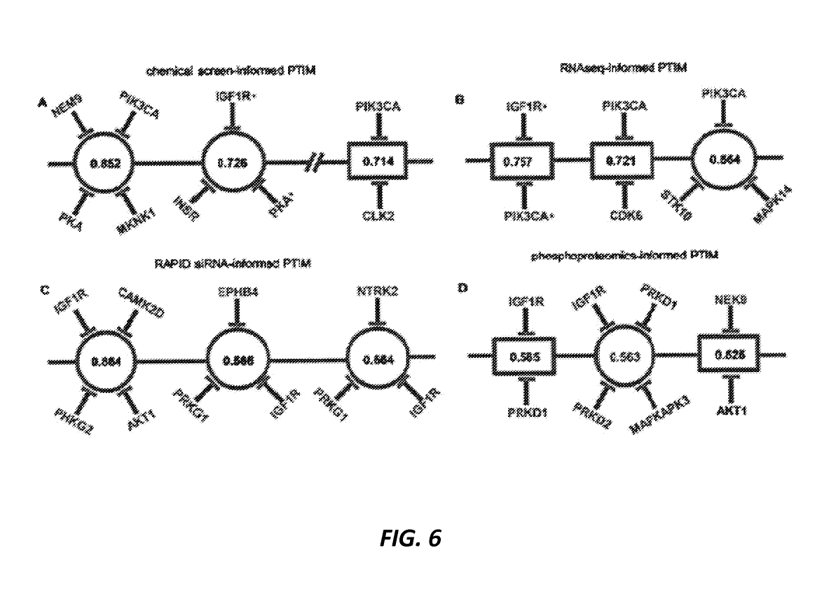

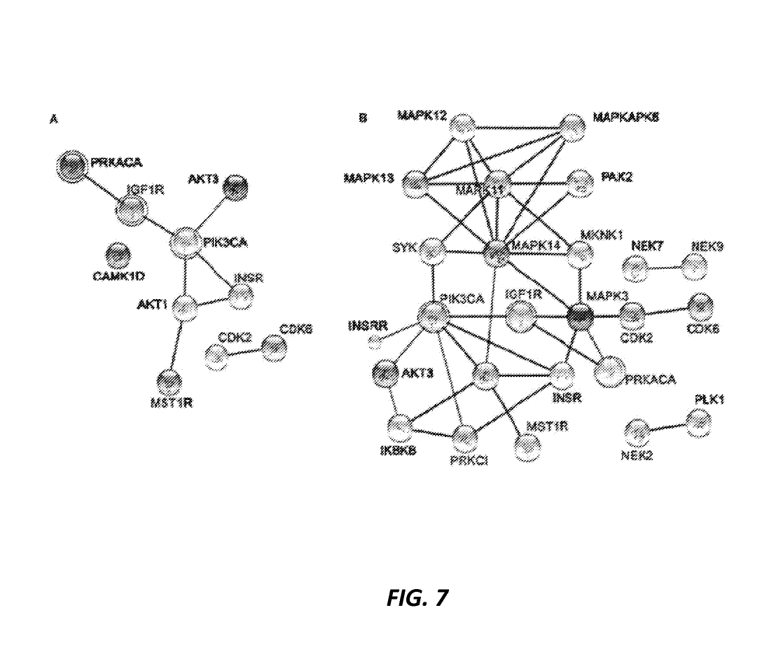

A final Roche-developed open access chemical screen consisting of 223 novel kinase inhibitor compounds (25) was also tested on U23674. The results of the chemical screen and associated modeling are provided in FIGS. 5 and 6.

To screen the chemical agents, cell cultures were plated in 384 well plates at a seeding density of 2000 cells per well over graded concentrations of the numerous compounds. The chemical concentrations were between 10 nM and 10004 depending on the activity range of the compounds. The cells were incubated in DMEM supplemented with 1% penicillin streptomycin, and 10% fetal bovine serum for 72 hours. Chemical sensitivity was determined by an MTS cell viability assay after the 72 hour incubation period. The sensitivity responses at the different concentrations were used to determine the chemical IC50 using a hill curve fitting algorithm with variable hill slope coefficients.

### Drug Combination Studies and Calculation of Combination Index (C.I.).

Drug combinations used in combination screens were guided by the models generated from the GlaxoSmithKline drug screen dataset. The target combinations identified were validated using compounds external to the drug screen dataset, and were selected based on the compounds primary putative targets. Single agent screens were initially performed to calculate independent drug efficacy as a preliminary step to combination experiments. All compounds were tested at dosages in the range 5 nM to 100 μM to bracket IC50 and IC25 dosage values. To perform the combination experiments, the IC25 dosage for the individual compound was tested in combination with gradated dosages of the complementary compound. Both compounds in a combination experiment were alternately tested as the fixed and gradated dosage compound. In addition, both compounds were retested as a single agent cohort to facilitate Combination Index (CI) quantification. To verify the mechanisms of action producing the desired synergistic effect in the combination experiments, a second set of validation experiment were performed. Here, low dosages of the combination agent were used based on the known EC50 values for the target of interest. In particular, a dosage equal to 5 times the EC50 value for the predicted target was used to validate the mechanism of synergy. Both agents in each combination were tested to verify the presence of synergy. The presence of synergy in these tests supports the mechanisms of action being the predicted targets. CI values were generated using CALCUSYN.

### RNA Deep Sequencing.

RNA sequencing was performed on the test sample U23674 and on the control sample consisting of regenerating mouse muscle tissue following cardiotoxin injury in vivo. The paired end raw reads for all RNAseq data was aligned using TopHat version 2.0.9. Up to two mismatches in the alignment were permitted before a read alignment was discarded. The reads were aligned to the UCSC mm19 reference mouse genome. The aligned reads were assembled into transcripts using CUFFLINKS version 2.1.1. The alignment quality and distribution of the reads were estimated via SAM tools version 0.1.18. compounds external to the drug screen dataset, and were selected based on the compounds primary putative targets. Single agent screens were initially performed to calculate independent drug efficacy as a preliminary step to combination experiments. All compounds were tested at dosages in the range 5 nM to 100 μM to bracket IC50 and IC25 dosage values. To perform the combination experiments, the IC25 dosage for the individual compound was tested in combination with gradated dosages of the complementary compound. Both compounds in a combination experiment were alternately tested as the fixed and gradated dosage compound. In addition, both compounds were retested as a single agent cohort to facilitate Combination Index (CI) quantification. To verify the mechanisms of action producing the desired synergistic effect in the combination experiments, a second set of validation experiment were performed. Here, low dosages of the combination agent were used based on the known EC50 values for the target of interest. In particular, a dosage equal to 5 times the EC50 value for the predicted target was used to validate the mechanism of synergy. Both agents in each combination were tested to verify the presence of synergy. The presence of synergy in these tests supports the mechanisms of action being the predicted targets. CI values were generated using CALCUSYN.

### RNA Deep Sequencing.

RNA sequencing was performed on the test sample U23674 and on the control sample consisting of regenerating mouse muscle tissue following cardiotoxin injury in vivo. The paired end raw reads for all RNAseq data was aligned using TOPHAT version 2.0.9. Up to two mismatches in the alignment were permitted before a read alignment was discarded. The reads were aligned to the UCSC mm19 reference mouse genome. The aligned reads were assembled into transcripts using CUFFLINKS version 2.1.1. The alignment quality and distribution of the reads were estimated via SAM tools version 0.1.18. The de novo merged transcript assembly from the aligned reads was performed via CUFFLINKS version 2.1.1. Differential comparison between the tumor sample and the control sample was performed by the Cuffdiff function of CUFFLINKS. For the differential analysis of tumor vs. control, the standard cuffdiff parameters were used. This was used to inform the differentially activated process analysis. It is important to note that for the integrative analysis that makes up the primary focus of this paper, we use as an indicator of overexpression an expression level in the tumor 50% greater than that in the matched normal sample.

RAPID siRNA Screen.

To assess the contribution of receptor tyrosine kinases to survival of the alveolar rhabdomyosarcoma sample, RAPID siRNA knockdown screening was performed of a primary cell culture of U23674. As previously described, 85 members of the mouse tyrosine kinase family were screened for single target efficacy. Target sensitivity was determined by resulting cell viability and was quantified using an MTS assay. Targets with viability two standard deviations below the mean were identified as high importance targets.

### Phosphoproteomic Screen.

To identify differentially phosphorylated protein targets, Phosphoproteomics Assays (Kinexus, Vancouver, British Columbia, Canada) were used to compare two duplicate cell lysates from U23674 against two duplicate cell lysates from regenerating muscle tissue acting as a control, and single lysates from two biologically-independent rhabdomyosarcoma samples (U48484 and U57810). To perform the phosphoproteomics analyses, 50 μg of lysate protein from each sample was covalently labeled with a proprietary fluorescent dye. Free dye molecules were removed by gel filtration. After blocking non-specific binding sites on the array, an incubation chamber was mounted onto the microarray to permit the loading of related samples side by side on the same chip. Following sample incubation, unbound proteins were washed away. Each array produces a pair of 16-bit images, which are captured with a Perkin-Elmer ScanArray Reader laser array scanner. Signal quantification was performed with IMAGENE 8.0 from BioDiscovery with predetermined settings for spot segmentation and background correction. The background-corrected raw intensity data are logarithmically transformed. Z scores are calculated by subtracting the overall average intensity of all spots within a sample from the raw intensity for each spot, and dividing it by the standard deviations (SD) of all of the measured intensities within each sample.

### Integrative Nonlinear Boolean Modeling

For analysis of the chemical screen data we use a proprietary nonlinear Boolean modeling approach called TIM (Target Inhibitor Map) modeling. PTIM is a predictive modeling approach to tumor drug sensitivity prediction based on inference of Boolean Relationships. The exemplary approach considers that the underlying mechanism for sensitivity to targeted drugs consists of a combination of series (inhibiting any one target in the series will block tumor proliferation and thus this behavior is similar to Boolean ‘OR’ logic) and parallel (all the parallel targets need to be inhibited to block tumor proliferation and thus this behavior is similar to Boolean ‘AND’ logic) targets being inhibited. For estimating the series and parallel targets, the response to multi-targeted drugs is analysed with both shared and separate targets. For instance, drugs having the same selective target (such as pelitinib and erlotinib, which are high selectivity drugs for kinase target EGFR) can show different sensitivity in vitro and this behavior can be attributed to the biologically relevant side targets of the drugs. The framework of the present example considers all possible targets of the drugs and generates abstract tumor proliferation pathways with most relevant targets that can best explain the drug screen responses. Here, secondary information is incorporated to refine the predicted models.

### RNAseq Integration.

For those targets that have both RNA expression data and presence in the drug screen, the expression data is utilized to eliminate possible false positives that could occur from drug screen results and to narrow down the true positives among relevant targets identified by the PTIM approach. The false positives are identified as the targets that are not highly expressed based on comparing the RNA expression of the cancerous cells to baseline expression values from a matched control sample. In this analysis, overexpression is determined as gene expression in the tumor sample 50% greater than that in the control sample. Note that expressed targets only are considered as the effect of a molecularly targeted drug is to inhibit the target when it is expressed and thus, non-expressed drug targets will have limited effect on predicting the drug response.

RAPID siRNA Screen Integration.

The RAPID screen results identify high sensitivity single target mechanisms of cancer cell growth inhibition. Targets identified in this manner were used as initial conditions for the sequential search portion of the model. Two models were generated in this way. The first model ensured the presence of the identified targets in the final target set. The second model allowed the targets to be selectively removed as the sequential search progressed. The representative model was selected based on the minimum error score of the two generated models.

### Kinexus Phosphoproteomics Screen.

The phosphoproteomics screen results identify differentially phosphorylated targets and associated pathways, Phosphorylation of these targets may be pushing the system towards a particular phenotype, and intervention in the form of changing phosphorylation status might result in significant changes to the system. Two models were generated in this way. The first model ensured the presence of the identified targets in the final target set. The second model allowed the targets to be selectively removed as the sequential search progressed. The representative model was selected based on the minimum error score of the two generated models.

### Orthotropic Allograft Studies.

SHO (SCID/hairless/outbred) mice (Charles River, Wilmington, Mass.) are orthotropically engrafted with 106 U23674 cells and mice treated with vehicle control (tartaric acid+TWEEN80/methylcellulose), 50 mg/kg OSI-906, 150 mg/kg GDC-0941, and combination 50 mg/kg OSI-906 plus 150 mg/kg GDC-0941. This engraftment was performed after damaging the right leg via cardiotoxin as previously described. Treatment commenced two days after engraftment. Dosing schedule was once daily by oral gavage up to day 5, at which time dosing was performed every other day. The endpoint for the study and survival analysis was tumor volume=1.4 cc.

### Resistance Abrogation Experiments.

This experiment utilizes pediatric and canine sarcoma specimens available through collaborations with the Oregon Health & Science University (OHSU) Pediatric Cancer Biology Program's Childhood Cancer Registry for Familial and Sporadic Tumors (CCuRe-FAST) and Oregon State University's (OSU) College of Veterinary Medicine. OHSU IRB and OSU IACUC approval was obtained. Primary cultures were established and cells were screened for sensitivity via the PPTI screen. The drug screen data was analyzed via the TIM modeling approach and key targets appearing in series were identified. The series target blocks indicate different treatments which may be effective.

Identifying two treatments working via independent pathways would indicate parallel treatment options. Therefore, if the tumor cells become resistant to one drug, the second drug will still act on another pathway and provide an opportunity to abrogate resistance. After TIM models were generated, a proof-of-principle experiment was undertaken to show that targets inhibited in series abrogate tumor cell resistance. Consensus targets were chosen for their appearance across TIM models in both species. Two drugs were chosen that most effectively inhibited targets in series at clinically achievable concentrations. A six arm in vitro trial was then undertaken in a representative canine and human sample. Each arm was performed in quadruplicate on 6-well plates with 10,000 cells per well. Cells were plated 24 hours prior to the administration of any drug. The drug concentration chosen were 1.5 times the Kd of the TIM target of interest. The drug selection was based on desired targets, as well as requiring drug concentration for reaching 1.5 times target Kd must also be less than the maximum clinically achievable concentration. The study arms were as follows: Arm 1—Drug 1 alone for 6 days; Arm 2—Drug 2 alone for 6 days; Arm 3—Drug 1 for 3 days, wash, then Drug 2 for 3 days; Arm 4-Drug 2 for 3 days, wash, then Drug 1 for 3 days; Arm 5—Drug 1 plus Drug 2 simultaneously for 6 days; Arm 6—vehicle control for 6 days. After the study period, each well was washed with Phosphate Buffered Saline (Gibco, Grand Island, N.Y.) and fresh DMEM with 10% FBS was placed in each well. Wells were monitored until confluency was observed. Primary study endpoint was days to well confluency as determined by a single user. Cells were also counted manually with a hemocytometer and photographed to confirm consistency of the user's definition of confluency. If after 100 days the cells did not reach confluency, the cell study would end and the remaining cells are counted. The experimental design and results are available in FIG. 11.

Supplemental Table 3

| | IC50 Value |
|---|---|
| Drug Name | (nM) |
| panobinostat (LBH-589) | 245 |
| bortezomib | 821 |
| carfilzomib | 860 |
| AUY922 | 1030 |
| BMS-754807 | 1730 |
| entinostat (SNDX-275, MS-275) | 1789 |
| vorinostat (SAHA) | 1967 |
| ABT-737 | 2659 |
| vandetanib (ZD6474, Zactima) | 3038 |
| quinacrine | 4915 |
| crizotinib (PF-2341066) | 5138 |
| fostamatinib (R788) | 5150 |
| MK-2206 | 5493 |
| trichostatin A | 5521 |
| sorafenib | 6128 |
| BIX 01294 | 6806 |
| GANT61 | 7378 |
| barasertib (AZD1152-HQPA) | >conc tested |
| decitabine (5-azacytadine) | >conc tested |
| enzastaurin | >conc tested |
| lapatinib | >conc tested |
| alisertib (MLN8237) | >conc tested |
| NAC | >conc tested |
| pazopanib (GW-786034) | >conc tested |
| ponatinib (AP24534) | >conc tested |
| RO4929097 | >conc tested |
| ruxolitinib (INCB018424) | >conc tested |
| selumetinib (AZD6244) | >conc tested |
| SJ-172550 | >conc tested |
| sodium butyrate | >conc tested |
| SP600125 | >conc tested |
| temsirolimus (CCI-779) | >conc tested |
| veliparib (ABT-888) | >conc tested |
| vismodegib (GDC-0449) | >conc tested |
| zibotentan (ZD4054) | >conc tested |
| OSI-906 | >conc tested |
| pelitinib (EKB-569) | >conc tested |
| tozasertib (VX-680) | >conc tested |
| obatoclax (GX15-070) | >conc tested |
| cediranib (AZD2171) | >conc tested |
| dasatinib | >conc tested |
| SNS-032 (BMS-387032) | >conc tested |
| alvocidib (Flavopiridol, NSC | >conc tested |
| 649890) | |
| AZD-8931 | >conc tested |
| BEZ235 | >conc tested |
| cabozantinib (XL-184) | >conc tested |
| cilengitide (EMD 121974) | >conc tested |
| dinaciclib (SCH-727965 | >conc tested |
| fenretinide (4-HPR) | >conc tested |
| lenalidomide (Revlimid, CC-5013) | >conc tested |
| nilotinib (AMN-107) | >conc tested |
| PF-562271 | >conc tested |
| tretinoin/vesanoid (ATRA) | >conc tested |
| nutlin-3 (RG7112, RO5045337 | >conc tested |
| saracatinib (AZD0530) | >conc tested |
| SB-431542 | >conc tested |
| tamoxifen (4-hydroxy-tamoxifen) | >conc tested |
| vemurafenib (PLX4032) | >conc tested |
| VER-155008 | >conc tested |
| SJ-172550 | >conc tested |

### Example 3

### Predictive Analysis to Identify Relevant Targets for Therapeutic Intervention

Provided herein are methodologies and results for a computational model of drug sensitivity designed to identify relevant functional targets in DIPG utilizing the Probabilistic Target Inhibition Map (PTIM) approach to generate multivariate blocks of targets whose joint inhibition is predicted to increase drug sensitivity. This approach incorporates only functional drug screen sensitivity values with available RNAseq expression data to identify key functional targets in the disease cohort.

### Data Pre-Processing and Notations.

For this analysis, a total of 6 samples were considered that had both drug sensitivity and RNA expression data. A subset consisting of 8 cell lines was tested on Drug Screen 1 (SU-DIPG-IV, SU-DIPG-VI, VU-DIPG.A, NEM-145, NEM157) and another subset of 7 cell lines was tested on Drug Screen 2 (NEM-163, NEM-165, NEM-168, NEM-175, JHH-DIPG1, NEM-157, NEM-215) with 2 cell lines tested on both screens.

To identify the mechanistic targets through which drugs derive their sensitivity, prior information on the kinase targets of the compounds are utilized. The Drug target inhibition information is contained in a matrix A which quantifies the ability of a drug to inhibit its kinase targets. Each row vector is relatively sparse, as most drugs inhibit a select few primary targets and a small set of secondary, but often relevant, side targets. Each entry of A is a real number between 0 and 1 denoting the inhibition of the kinase target. An entry of A is close to 1 when a small concentration of a drug can phosphorylate 50% of the kinase target. The matrix A is generated based on published drug target Kd's or EC50's from http://pubchem.ncbi.nlm.nih.gov/ and related publications. A hill curve interpolation is used with hill coefficient of 1 and drug concentration of 10,000 nM to convert the EC50's to normalized inhibitions between 0 and 1. For each drug tested over a cell line, the functional response is measured as cell viability, the percentage of living cancerous cells following 72 hour application of a drug. The cellular death rate for each drug, considered as the percentage of cancerous cells killed following 72-hour application of the drug, is simply (1−cell viability). The cellular death rate values are tested with 3 replicates in 4 concentrations. The cellular death rate values are converted to IC50's via hill curve interpolation. The IC50 values are then converted to sensitivities between 0 and 1 using a hill curve with hill coefficient of 1 and drug concentration of 10,000 nM. A NaN or NA IC50 is converted to sensitivity 0. This set of sensitivities is a matrix denoted by Σ. Note that we have a matrix Σ of size 60×8 for drug screen 1 and a matrix Σ of size 60×7 for drug screen 2.

To incorporate the mutation characterization, we consider the set of kinase targets present in the drug screen (denoted by Θ₁) and created a binary matrix M₀ which denotes whether each of the drug target is mutated or not for each cell line. Considering the common 404 targets of the two drug screens, the size of M₀ is 15×404 where 15 denotes the cell lines (two rows are repeated as there are unique 10 cell lines).

To incorporate RNA expression data generated from the RNAseq experiments, we consider the set of kinase targets present in the drug screen (denoted Θ₁) and the targets with quantified expression present in the RNAseq data (denoted Θ₂). The set of targets for which we have usable information is then Θ₁∩Θ₂, the intersection of the two target sets. The remaining targets in Θ₁ are targets for which we do not have any RNA expression information and thus we are only able to gain information on these kinase targets from the drug screen data. Let the RNA expression be given by matrix G₀. In this analysis, the set Θ₁∩Θ₂ has 403 targets and thus the size of G₀ is 15×403. The RNA expression matrix is converted to a normalized matrix G by dividing the RNA expression of each gene by the mean expression of the matched normal samples or mean expression values. Thus a value >>1 in G denotes that the gene is highly expressed for that sample compared to normal matched samples or the expression mean. TIM predictive algorithm to identify multivariate target combinations for therapeutic intervention.

In this section, the Probabilistic Target Inhibition Map approach is used for target selection and sensitivity prediction. Next is providing a brief outline of the approach. The primary goal of the PTIM model is to generate hypotheses of set of targets that can potentially produce desirable treatment results based on the available drug screen and gene expression data.

There are two main steps in the PTIM method: step one is selection of the optimal drug targets based on functional data generated from drug screens. This is the model generation step in the PTIM algorithm. The model generation is approached from a Boolean logic perspective, where logical relationships are generated between targets that identify the mechanisms by which tumors persist. Due to the Boolean nature of the approach, the matrix A generated for this analysis consists of 0's or 1's. The drug inhibition profiles (in the form of target EC₅₀'s) are used to generate the binarized inhibition vectors based on the drug's IC50 value. The binarized vectors are generated based on the following equation: a log(IC₅₀)<log(EC₅₀)<b log(IC₅₀), 0≦a<<b This binarization approach uses the functional drug sensitivity data to generate vectors of relevant drug targets. It is assumed that any target sufficiently beyond the IC₅₀ point is unlikely to have been the cause of the drug response, and thus information cannot be from it.

To generate drug sensitivity values from the IC₅₀ response data, the following equation:

$${y}_{i} = \frac{log ( {MaxDose}_{i} ) - log ( {IC}_{50 , i} )}{log ( {MaxDose}_{i} )}$$

is used, where IC_{50,i} is the IC_{50,i} of drug i, MaxDoseᵢ is the maximum tested dosage of drug i, and yᵢ is the resulting sensitivity value of drug i.

To incorporate RNAseq data into the PTIM analysis, unlikely kinase targets are eliminated from consideration based on G, the matrix of normalized RNA expressions over the set Θ₁∩Θ₂. A kinase target is kept in the dataset for consideration if G(i,j)>p, a threshold for minimum relative expression. Note that expressed targets are considered only as the effect of a molecularly-targeted drug is to inhibit the target when it is expressed and thus, non-expressed drug targets will have limited effect on predicting the drug response. For the purpose of this project, the RNA expressions constraint was G(i,j)>ρ=1 meaning the RNA expression of the cancerous line must be at least that of the matched normal expression. As G does not provide information about the remaining targets in Θ₁-Θ₂, they are not eliminated from consideration as they could potentially be driver mechanisms for the tumor. primary analysis is performed on the 16 sets of joint RNAseq-matched drug screen data and cell line drug screen data.

### Subset-Superset Biological Constraint.

The following biological constraints are utilized to identify consistent and inconsistent sets of protein targets². For any drug d₁ and any kinase target set S_{T}, Constraint 1: If Δ(d₁, S_{T}) [symbol] Δ(d₂, S_{T}), then Σ(i, d₁)≦Σ(i, d₂) (more inhibition of oncogenes should improve sensitivity) Constraint 2: If Δ(d₁, S_{T}) [symbol] Δ(d₂, S_{T}), then Σ(i, d₁)≧Σ(i, d₂) (less inhibition of oncogenes will reduce sensitivity)

Here, the [symbol] , [symbol] operators signify component-wise inequality in all components. The vectors and associated sensitivity values from matrix Σ are used to select the optimal targets for the PTIM model. The optimal targets are selected by solving the following optimization problem that incorporates the inter-bin error (the error associated with multiple drugs having the same inhibition profile but differences in their sensitivity values) and the inconsistency error (the error associated with drug A being less effective than drug B but the targets of drug B are a subset of the drug A's targets). In this context, a bin is any subset of a set of kinase targets selected to build the model. The equation for inter-bin error is given by Σ_{bins}Σ_{jεbin}|P(sⱼ|T)−Y(sⱼ|T)|, where P(•) is expected sensitivity of sⱼ under target set T, and Y(•) is the experimental sensitivity of sⱼ under target set T. The inter-bin error amounts to ensuring that kinase inhibitors with similar inhibition profiles with respect to the model have similar experimental sensitivities, indicating the likely mechanisms have been identified. The inconsistency error incorporates the subset-superset biological constraint introduced previously and is given by the following equation: Σ_{drugs}Σ_{bins}χ(bin, drug)|P(sⱼ|T)−Y(sⱼ|T)|, where P(•) and Y(•) are as above and χ(•) is an indicator function which is 1 when the subset-superset constraint is violated. The total optimization problem then is:

Σ_{bins}Σ_{jεbin}|P(sⱼ|T)−Y(sⱼ|T)|+Σ_{drugs}Σ_{bins}χ(bin,drug)|P(sⱼ|T)−Y(sⱼ|T)|.

The minimum solution to the optimization problem using a suboptimal search algorithm known as Sequential Floating Forward Search (SFFS). The PTIM framework requires that the subset-superset biological constraints are satisfied during model generation. This allows for identification of unique target combinations that may not be identified as relevant through a standard linear modeling approach.

Step two in PTIM analysis is sensitivity prediction based on selected targets from step one. This step is used to generate the PTIM circuit, which provides a visual representation of effective treatments inferred from the functional data. To generate the target blocks, viable target combinations are explored systematically in a breadth-first search style approach. Here, desired sensitivity levels for each level of inhibition (in terms of targets inhibited) is specified by the user; based on this specification, combinations of targets that satisfy the desired sensitivity levels are selected and no further supersets of the combination are considered. The inferential steps are based on bounding the expected sensitivity by the closest subset of the target combination and the closest superset of the target combination. The equation for inferred sensitivity for a target set {t₁, t₂, . . . tₙ}. is given by

$$y ( \{ {t}_{1} , {t}_{2} , … , {t}_{n} \} ) = {y}_{l} + ( {y}_{u} - {y}_{l} ) {( \frac{{\sum }_{i = 1}^{n} α ( {t}_{i} )}{{\sum }_{i = 1}^{h} α ( {t}_{i} )} )}^{β} ,$$

where yᵢ is the maximum sensitivity of the subsets of {t₁, t₂, . . . tₙ}, yᵤ is the minimum sensitivity of the supersets of {t₁, t₂, . . . tₙ}, α(•) is weight for a kinase target, and β determines the order of the curve fit for y({t₁, t₂, . . . , tₙ}). For this analysis, a(•)=1 for all targets meaning all targets have equal weight and β=1 which provides a linear fit. The sum in the denominator is the sum of weights between the subset generating yᵢ and the superset generating yᵤ. In this way, the predicted sensitivity lies between y₁ and yᵤ depending on the distance of {t₁, t₂, . . . , tₙ} to the subset and superset. Based on the number of additional targets needed to move from the subset to the desired target combination, the sensitivity of the drug combination is inferred. In the event, the predicted sensitivity y({t₁, t₂, . . . , tₙ}) is sufficient to satisfy the sensitivity level defined by the user, the target combination is designated a valid block combination, and the supersets are no longer considered.

Note that PTIM analysis generates potential targets from drug screen information and uses the gene expression to remove false positives. Since the drugs considered inhibit oncogenes, we keep the targets that are differentially expressed in tumor cell lines as compared to normal cell lines. If a PTIM analysis target is differentially expressed in at least one tumor cell line, the target is considered relevant. The number of genes displayed belongs to blocks whose scores are above a threshold of 0.5.

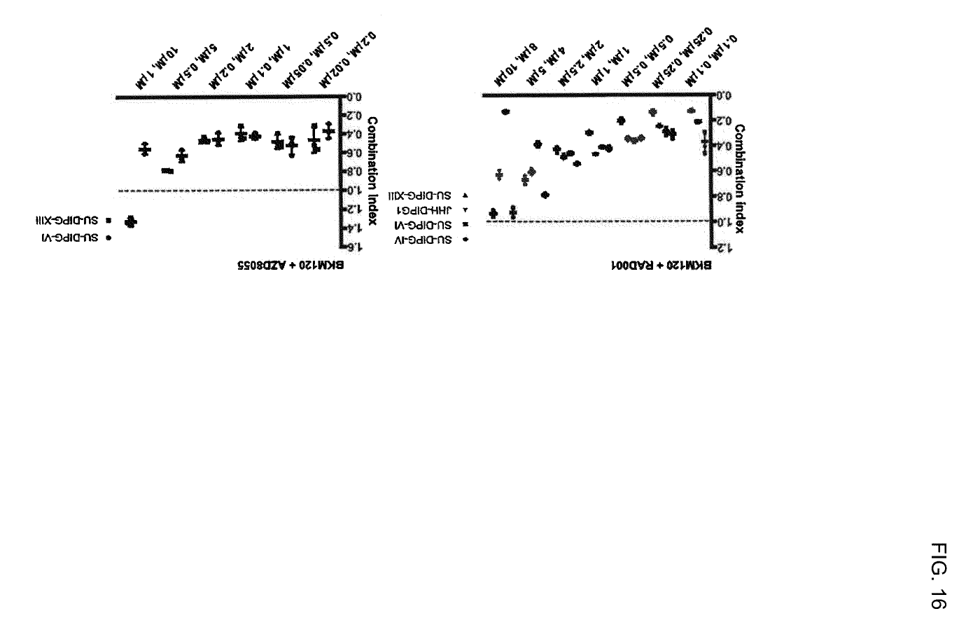

FIG. 16 contains the results of in vitro drug combination testing and synergy analysis for a target combination predicted by the TIM modeling approach to have in vitro synergy. The Chou-Talalay Combination Index (CI) was used to detect synergy (CI<1). The CI is calculated based on the following equation:

$$C I = \frac{{D}_{1}}{{D}_{x 1}} + \frac{{D}_{2}}{{D}_{x 2}}$$

where D₁ and D₂ denotes the combination dosages tested for drugs 1 and 2 to achieve 6% inhibition and Dₓ₁ and Dₓ₂ denote the corresponding dosages of individuals drugs 1 and 2 required to inhibit δ %. To provide quantification for the predictive power of the TIM approach, 10-fold cross validation mean absolute errors (MAE) and correlation coefficients between experimental and predictive sensitivities were calculated. The first test used only the functional data generated from the Drug Screens for prediction. For this analysis the 10-fold CV error was 0.101 and a correlation coefficient of 0.848. The second test incorporated the RNA expression data to eliminate potential false positive before model development. Here the 10-fold CV error was 0.111 and correlation coefficient of 0.773.

Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently. While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

### REFERENCES

1. Kohler B A, Ward E, McCarthy B J, Schymura M J, Ries L A, et al. (2011) Annual report to the nation on the status of cancer, 1975-2007, featuring tumors of the brain and other nervous system. Journal of the National Cancer Institute 103: 714-736.
2. Ries L A G, Smith M A, Gurney J G, Linet M, Tamra T, et al. (1999) Cancer Incidence and Survival among Children and Adolescents: United States SEER Program 1975-1995. NIH Pub. No. 99-4649.
3. Minturn J E, Fisher M J (2013) Gliomas in children. Curr Treat Options Neurol 15: 316-327.
4. Freeman C R, Farmer J P (1998) Pediatric brain stem gliomas: a review. International journal of radiation oncology, biology, physics 40: 265-271.
5. Berger M S, Edwards M S, LaMasters D, Davis R L, Wilson C B (1983) Pediatric brain stem tumors: radiographic, pathological, and clinical correlations. Neurosurgery 12: 298-302.
6. Littman P, Jarrett P, Bilaniuk L T, Rorke L B, Zimmerman R A, et al. (1980) Pediatric brain stem gliomas. Cancer 45: 2787-2792.
7. Hargrave D, Bartels U, Bouffet E (2006) Diffuse brainstem glioma in children: critical review of clinical trials. The lancet oncology 7: 241-248.
8. Freeman C R, Farmer J-P (1998) Pediatric brain stem gliomas: A review. International Journal of Radiation Oncology*Biology*Physics 40: 265-271.
9. Roujeau T, Machado G, Garnett M R, Miguel C, Puget S, et al. (2007) Stereotactic biopsy of diffuse pontine lesions in children. Journal of neurosurgery 107: 1-4.
10. Khuong-Quang D A, Buczkowicz P, Rakopoulos P, Liu X Y, Fontebasso A M, et al. (2012) K27M mutation in histone H3.3 defines clinically and biologically distinct subgroups of pediatric diffuse intrinsic pontine gliomas. Acta Neuropathol 124: 439-447.
11. Warren K E (2012) Diffuse intrinsic pontine glioma: poised for progress. Front Oncol 2: 205.
12. Grill J, Puget S, Andreiuolo F, Philippe C, MacConaill L, et al. (2012) Critical oncogenic mutations in newly diagnosed pediatric diffuse intrinsic pontine glioma. Pediatr Blood Cancer 58: 489-491.
13. Paugh B S, Broniscer A, Qu C, Miller C P, Zhang J, et al. (2011) Genome-wide analyses identify recurrent amplifications of receptor tyrosine kinases and cell-cycle regulatory genes in diffuse intrinsic pontine glioma. J Clin Oncol 29: 3999-4006.
14. Puget S, Philippe C, Bax D A, Job B, Varlet P, et al. (2012) Mesenchymal transition and PDGFRA amplification/mutation are key distinct oncogenic events in pediatric diffuse intrinsic pontine gliomas. PLoS One 7: e30313.
15. Zarghooni M, Bartels U, Lee E, Buczkowicz P, Morrison A, et al. (2010) Whole-genome profiling of pediatric diffuse intrinsic pontine gliomas highlights platelet-derived growth factor receptor alpha and poly (ADP-ribose) polymerase as potential therapeutic targets. J Clin Oncol 28: 1337-1344.
16. Albright A L, Packer R J, Zimmerman R, Rorke L B, Boyett J, et al. (1993) Magnetic resonance scans should replace biopsies for the diagnosis of diffuse brain stem gliomas: a report from the Children's Cancer Group. Neurosurgery 33: 1026-1029; discussion 1029-1030.
17. Becher O J, Hambardzumyan D, Walker T R, Helmy K, Nazarian J, et al. (2010) Preclinical evaluation of radiation and perifosine in a genetically and histologically accurate model of brainstem glioma. Cancer Res 70: 2548-2557.
18. Monje M, Mitra S S, Freret M E, Raveh T B, Kim J, et al. (2011) Hedgehog-responsive candidate cell of origin for diffuse intrinsic pontine glioma. Proc Natl Acad Sci USA 108: 4453-4458.
19. Nakamura S, Watanabe H, Miura M, Sasaki T (1997) Effect of the insulin-like growth factor I receptor on ionizing radiation-induced cell death in mouse embryo fibroblasts. Exp Cell Res 235: 287-294.
20. Turner B C, Haffty B G, Narayanan L, Yuan J, Havre P A, et al. (1997) Insulin-like growth factor-I receptor overexpression mediates cellular radioresistance and local breast cancer recurrence after lumpectomy and radiation. Cancer Res 57: 3079-3083.
21. Wojtalla A, Salm F, Christiansen D G, Cremona T, Cwiek P, et al. (2012) Novel agents targeting the IGF-1R/P I3K pathway impair cell proliferation and survival in subsets of medulloblastoma and neuroblastoma. PLoS One 7: e47109.
22. Yin S, Girnita A, Stromberg T, Khan Z, Andersson S, et al. (2010) Targeting the insulin-like growth factor-1 receptor by picropodophyllin as a treatment option for glioblastoma. Neuro Oncol 12: 19-27.
23. Resnicoff M, Rubini M, Baserga R, Rubin R (1994) Ethanol inhibits insulin-like growth factor-1-mediated signalling and proliferation of C6 rat glioblastoma cells. Lab Invest 71: 657-662.
24. Resnicoff M, Sell C, Rubini M, Coppola D, Ambrose D, et al. (1994) Rat glioblastoma cells expressing an antisense RNA to the insulin-like growth factor-1 (IGF-1) receptor are nontumorigenic and induce regression of wild-type tumors. Cancer Res 54: 2218-2222.
25. Scotlandi K, Avnet S, Benini S, Manara M C, Serra M, et al. (2002) Expression of an IGF-I receptor dominant negative mutant induces apoptosis, inhibits tumorigenesis and enhances chemosensitivity in Ewing's sarcoma cells. Int J Cancer 101: 11-16.
26. Huang F, Hurlburt W, Greer A, Reeves K A, Hillerman S, et al. (2010) Differential mechanisms of acquired resistance to insulin-like growth factor-i receptor antibody therapy or to a small-molecule inhibitor, BMS-754807, in a human rhabdomyosarcoma model. Cancer Res 70: 7221-7231.
27. Kolb E A, Gorlick R, Lock R, Carol H, Morton C L, et al. (2011) Initial testing (stage 1) of the IGF-1 receptor inhibitor BMS-754807 by the pediatric preclinical testing program. Pediatr Blood Cancer 56: 595-603.
28. Barton K L, Misuraca K, Cordero F, Dobrikova E, Min H D, et al. (2013) PD-0332991, a CDK4/6 inhibitor, significantly prolongs survival in a genetically engineered mouse model of brainstem glioma. PLoS One 8: e77639.
29. Lewis P W, Muller M M, Koletsky M S, Cordero F, Lin S, et al. (2013) Inhibition of PRC2 activity by a gain-of-function H3 mutation found in pediatric glioblastoma. Science 340: 857-861.
30. Berlow N, Davis L E, Cantor E L, Seguin B, Keller C, et al. (2013) A new approach for prediction of tumor sensitivity to targeted drugs based on functional data. BMC Bioinformatics 14: 239.
31. Pal R, Berlow N (2012) A kinase inhibition map approach for tumor sensitivity prediction and combination therapy design for targeted drugs. Pac Symp Biocomput: 351-362.
32. Maximova O A, Taffs R E, Pomeroy K L, Piccardo P, Asher D M (2006) Computerized morphometric analysis of pathological prion protein deposition in scrapie-infected hamster brain. J Histochem Cytochem 54: 97-107.
33. Lee J, Kotliarova S, Kotliarov Y, Li A, Su Q, et al. (2006) Tumor stem cells derived from glioblastomas cultured in bFGF and EGF more closely mirror the phenotype and genotype of primary tumors than do serum-cultured cell lines. Cancer Cell 9: 391-403.
34. Bielen A, Perryman L, Box G M, Valenti M, de Haven Brandon A, et al. (2011) Enhanced efficacy of IGF1R inhibition in pediatric glioblastoma by combinatorial targeting of PDGFRalpha/beta. Mol Cancer Ther 10: 1407-1418.
35. Druker B J. Translation of the Philadelphia chromosome into therapy for CML. Blood. 2008; 112(13):4808-17.
36. Green M R. Targeting Targeted Therapy. New England Journal of Medicine. 2004; 350(21):2191-3.
37. Hopkins A L, Mason J S, and Overington J P. Can we rationally design promiscuous drugs? Current opinion in structural biology. 2006; 16(1):127-36.
38. Sos M L, Michel K, Zander T, Weiss J, Frommolt P, Peifer M, Li D, Ullrich R, Koker M, Fischer F, et al. Predicting drug susceptibility of non-small cell lung cancers based on genetic lesions. The Journal of Clinical Investigation. 2009; 119(6):1727-40.
39. Staunton J E, Slonim D K, Coller H A, Tamayo P, Angelo M J, Park J, Scherf U, Lee J K, Reinhold W O, Weinstein J N, et al. Chemosensitivity prediction by transcriptional profiling. Proceedings of the National Academy of Sciences. 2001; 98(19):10787-92.
40. Garnett M J, Edelman E J, Heidorn S J, Greenman C D, Dastur A, Lau K W, Greninger P, Thompson I R, Luo X, Soares J, et al. Systematic identification of genomic markers of drug sensitivity in cancer cells. Nature. 2012; 483(7391):570-5.
41. Barretina J, Caponigro G, Stransky N, Venkatesan K, Margolin A A, Kim S, Wilson C J, Lehar J, Kryukov G V, Sonkin D, et al. The Cancer Cell Line Encyclopedia enables predictive modelling of anticancer drug sensitivity. Nature. 2012; 483(7391):603-307.
42. Lee J K, Havaleshko D M, Cho H, Weinstein J N, Kaldjian E P, Karpovich J, Grimshaw A, and Theodorescu D. A strategy for predicting the chemosensitivity of human cancers and its application to drug discovery. Proceedings of the National Academy of Sciences. 2007; 104(32):13086-91.
43. Riddick G, Song H, Ahn S, Walling J, Borges-Rivera D, Zhang W, and Fine H A. Predicting in vitro drug sensitivity using Random Forests. Bioinformatics (Oxford, England). 2011; 27(2):220-4.
44. Wan Q, and Pal R. An Ensemble Based Top Performing Approach for NCI-DREAM Drug Sensitivity Prediction Challenge. PLoS ONE. 2014; 9(6):e101 183.
45. Costello J C, Heiser L M, Georgii E, Gonen M, Menden M P, Wang N J, Bansal M, Ammaduddin M, Hintsanen P, Khan S A, et al. A community effort to assess and improve drug sensitivity prediction algorithms. Nat Biotech. 2014; 32(12):1202-12.
46. Mitsos A, Melas I N, Siminelakis P, Chairakaki A D, Saez-Rodriguez J, and Alexopoulos L G. Identifying Drug Effects via Pathway Alterations using an Integer Linear Programming Optimization Formulation on Phosphoproteomic Data. PLoS Comput Biol. 2009; 5(12):e1000591.
47. Walther Z, and Sklar J. Molecular tumor profiling for prediction of response to anticancer therapies. Cancer journal (Sudbury, Mass.). 2011; 17(2):71-9.
48. Molinelli E J, Korkut A, Wang W, Miller M L, Gauthier N P, Jing X, Kaushik P, He Q, Mills G, Solit D B, et al. Perturbation Biology: Inferring Signaling Networks in Cellular Systems. PLoS Comput Biol. 2013; 9(12):e1003290.
49. Berlow N, Davis L E, Cantor E L, Seguin B, Keller C, and Pal R. A new approach for prediction of tumor sensitivity to targeted drugs based on functional data. BMC bioinformatics. 2013; 14(239.
50. Pal R, and Berlow N. A kinase inhibition map approach for tumor sensitivity prediction and combination therapy design for targeted drugs. Pacific Symposium on Biocomputing Pacific Symposium on Biocomputing. 2012:351-62.
51. Berlow N, Davis L, Keller C, and Pal R. Inference of dynamic biological networks based on responses to drug perturbations. EURASIP Journal on Bioinformatics and Systems Biology. 2014; 2014(1):14.
52. Berlow N, Haider S, Qian W, Geltzeiler M, Davis L E, Keller C, and Pal R. An Integrated Approach to Anti-Cancer Drug Sensitivity Prediction. Computational Biology and Bioinformatics, IEEE/ACM Transactions on. 2014; 11(6):995-1008.
53. Abraham J, Nunez-Alvarez Y, Hettmer S, Carrio E, Chen H I H, Nishijo K, Huang E T, Prajapati S I, Walker R L, Davis S, et al. Lineage of origin in rhabdomyosarcoma informs pharmacological response. Genes & Development. 2014; 28(14):1578-91.
54. Rubin B P, Nishijo K, Chen H I, Yi X, Schuetze D P, Pal R, Prajapati S I, Abraham J, Arenkiel B R, Chen Q R, et al. Evidence for an unanticipated relationship between undifferentiated pleomorphic sarcoma and embryonal rhabdomyosarcoma. Cancer cell. 2011; 19(2):177-91.
55. Aslam M I, Abraham J, Mansoor A, Druker B J, Tyner J W, and Keller C. PDGFRbeta reverses EphB4 signaling in alveolar rhabdomyosarcoma. Proceedings of the National Academy of Sciences of the United States of America. 2014; 111(17):6383-8.
56. Abraham J, Prajapati S I, Nishijo K, Schaffer B S, Taniguchi E, Kilcoyne A, McCleish A T, Nelon L D, Giles F G, Efstratiadis A, et al. Evasion mechanisms to Igflr inhibition in rhabdomyosarcoma. Molecular cancer therapeutics. 2011; 10(4):697-707.
57. Edwards A M, Bountra C, Kerr D J, and Willson™. Open access chemical and clinical probes to support drug discovery. Nat Chem Biol. 2009; 5(7):436-40.
58. Fedorov O, Muller S, and Knapp S. The (un)targeted cancer kinome. Nat Chem Biol. 2010; 6(3):166-9.
59. Goodnow R A, Jr., and Gillespie P. Hit and Lead identification: efficient practices for drug discovery. Progress in medicinal chemistry. 2007; 45(1-61.
60. Luk K C, Simcox M E, Schutt A, Rowan K, Thompson T, Chen Y, Kammlott U, DePinto W, Bioorganic & medicinal chemistry letters. 2004; 14(4):913-7.
61. Gong L, Hirschfeld D, Tan Y C, Heather Hogg J, Peltz G, Avnur Z, and Dunten P. Discovery of potent and bioavailable GSK-3beta inhibitors. Bioorganic & medicinal chemistry letters. 2010; 20(5):1693-6.
62. Davis P D, Elliott L H, Harris W, Hill C H, Hurst S A, Keech E, Kumar M K, Lawton G, Nixon J S, and Wilkinson S E. Inhibitors of protein kinase C. 2. Substituted bisindolylmaleimides with improved potency and selectivity. Journal of medicinal chemistry. 1992; 35(6):994-1001.
63. Davis P D, Hill C H, Lawton G, Nixon J S, Wilkinson S E, Hurst S A, Keech E, and Turner S E. Inhibitors of protein kinase C. 1. 2,3-Bisarylmaleimides. Journal of medicinal chemistry. 1992; 35(1):177-84.
64. Liu J J, Daniewski I, Ding Q, Higgins B, Ju G, Kolinsky K, Konzelmann F, Lukacs C, Pizzolato G, Rossman P, et al. Pyrazolobenzodiazepines: part I. Synthesis and SAR of a potent class of kinase inhibitors. Bioorganic & medicinal chemistry letters. 2010; 20(20):5984-7.
65. Trejo A, Arzeno H, Browner M, Chanda S, Cheng S, corner D D, Dalrymple S A, Dunten P, Lafargue J, Lovejoy B, et al. Design and synthesis of 4-azaindoles as inhibitors of p38 MAP kinase. Journal of medicinal chemistry. 2003; 46(22):4702-13.
66. Dermatakis A, Luk K C, and DePinto W. Synthesis of potent oxindole CDK2 inhibitors; Bioorganic & medicinal chemistry. 2003; 11(8):1873-81.
67. Goldstein D M, Alfredson T, Bertrand J, Browner M F, Clifford K, Dalrymple S A, Dunn J, Freire-Moar J, Harris S, Labadie S S, et al. Discovery of S-[5-amino-1-(4-fluorophenyl) 1Hpyrazol-4-yl]-[3-(2,3-dihydroxypropoxyl)phenyl]methanone (R03201195), an orally bioavailable and highly selective inhibitor of p38 MAP kinase. Journal of medicinal chemistry. 2006; 49(5):1562-75.
68. McDermott L A, Simcox M, Higgins B, Nevins T, Kolinsky K, Smith M, Yang H, Li J K, Chen Y, Ke J, et al. R04383596, an orally active KDR, FGFR, and PDGFR inhibitor: synthesis and biological evaluation. Bioorganic & medicinal chemistry. 2005; 13(16):4835-41.
69. Breitenlechner C B, Wegge T, Berillon L, Graul K, Marzenell K, Friebe W G, Thomas U, Schumacher R, Huber R, Engh R A, et al. Structure-based optimization of novel azepane derivatives as PKB inhibitors. Journal of medicinal chemistry. 2004; 47(6):1375-90.
70. Goldstein D M, Soth M, Gabriel T, Dewdney N, Kuglstatter A, Arzeno H, Chen J, Bingenheimer W, Dalrymple S A, Dunn J, et al. Discovery of 6-(2,4-difluorophenoxy)-2-[3-hydroxy-1-(2-hydroxyethyl)propylamino]-8-methyl-8H-pyrido[2,3-d]pyrimidin-7-one (pamapimod) and 6-(2,4-difluorophenoxy)-8-methyl-2-(tetrahydro-2H-pyran-4-ylamino)pyrido[2,3-d]pyrimidin-7(8H)-one (R1487) as orally bioavailable and highly selective inhibitors of p38alpha mitogen-activated protein kinase. Journal of medicinal chemistry. 2011; 54(7):2255-65.
71. Chu X J, DePinto W, Bartkovitz D, So S S, Vu B T, Packman K, Lukacs C, Ding Q, Jiang N, Wang K, et al. Discovery of [4-Amino-2-(1-methanesulfonylpiperidin-4-ylamino)pyrimidin-5-yl](2,3-difluoro-6-methoxyphenyl)methanone (R547), a potent and selective cyclin dependent kinase inhibitor with significant in vivo antitumor activity. Journal of medicinal chemistry. 2006; 49(22):6549-60

What is claimed is:

1. A method comprising:
(a) identifying a patient diagnosed with a cancer;
(b) generating a testable culture from the cancer;
(c) testing viability of the testable culture against one or more targeted drugs;
(d) generating, via a computing device, a probabilistic target inhibition map (PTIM) from the viability data that produces the lowest error in sensitivity prediction; wherein the computational algorithm involves (i) a selection of a set of targets that satisfies the biological constraint of increased inhibition of oncogenic targets increasing sensitivity, and (ii) generation of a probabilistic model based on the selected targets that produces high accuracy sensitivity prediction for unknown drugs with known target inhibition profile;
(e) generating data from one or more secondary screens of the cancer or the testable culture;
(f) creating, via a computing device, a genomics and proteomics informed PTIM (GPI-PTIM) based on the PTIM and the data from step (e);
(g) designing, via a computing device, a designed combination therapy based on the GPI-PTIM, wherein the GPI-PTIM is configured to yield higher sensitivity with minimal target inhibition or avoiding resistance to drugs by targeting multiple pathways; and
(h) validating the designed combination therapy in vitro against the testable culture to yield a validated combination therapy.

2. The method of claim 1, wherein the designing step (g) further comprises considering the selection of a set of drugs from available drugs using approximation algorithms, wherein the combined toxicity of the set of drugs is restricted by an upper bound and the sensitivity of the combination drugs is predicted to be synergistic.

3. The method of claim 1, wherein step (h) further comprises validating the combination therapy in an in vivo mouse xenograft model.

4. The method of claim 3, wherein the validated combination therapy will be the designed combination therapy that demonstrates the best activity against the cancer in vitro and in vivo.

5. The method of claim 1, further comprising: (i) repeating steps (c) to (h) if a validated combination therapy is not identified.

6. The method of claim 1, further comprising: (i) repeating steps (b) to (h) if a validated combination therapy is not identified.

7. The method of claim 1, further comprising: (j) treating the patient with the validated combination therapy.

8. The method of claim 1, wherein the secondary screens may be RNA sequencing, DNA sequencing, protein expression testing, histomorphology, or medical imaging.

9. The method of claim 8, wherein protein expression testing may comprise immunohistochemistry scoring.

10. The method of claim 8, wherein histomorphology may comprise round versus spindle cell feature scoring.

11. The method of claim 8, wherein medical imaging may comprise imaging cellular features such as shape, roundness, or the interdigitating roughness of an invasive tumor.

12. The method of claim 1, wherein the testable culture may be a single cell suspension, a primary cell culture, or a cell line established from the cancer of the patient.

13. A method comprising:
(a) receiving, at a computing device over a network from a user, a query comprising cell viability data associated with a testable culture of a patient's tumor;
(b) extracting, via the computing device, features of the cell viability data from the testable culture, said features comprising information associated with one or more targeted drugs;
(c) generating, via the computing device, a probabilistic target inhibition map (PTIM) from the viability data that produces the lowest error in sensitivity prediction; wherein the computational algorithm involves (i) a selection of a set of targets that satisfies the biological constraint of increased inhibition of oncogenic targets increasing sensitivity, and (ii) generation of a probabilistic model based on the selected targets that produces high accuracy sensitivity prediction for unknown drugs with known target inhibition profile;
(d) receiving, at a computing device, secondary data from one or more cancer screens from the testable culture;
(e) comparing, via the computing device, the PTIM of the cell viability data with the secondary data from one or more secondary screens from the testable culture, said comparison comprising creating a genomics and proteomics informed PTIM (GPI-PTIM);
(f) designing, via the computing device, a designed combination therapy based on the GPI-PTIM, wherein the GPI-PTIM is configured to yield higher sensitivity with minimal target inhibition or avoiding resistance to drugs by targeting multiple pathways; and
(f) communicating, via the computing device over the network, a designed combination therapy that may be validated to a validated combination therapy.

14. The method of claim 13, wherein the designing step (f) further comprises considering the selection of a set of drugs from available drugs using approximation algorithms via the computing device, wherein the combined toxicity of the set of drugs is restricted by an upper bound and the sensitivity of the combination drugs is predicted to be synergistic.

15. The method of claim 13, further comprising: (g) repeating steps (a) to (f) if a validated combination therapy is not identified.

16. A system comprising:
(a) a processor; and
(b) a non-transitory computer-readable storage medium for tangibly storing thereon program logic for execution by the processor, the program logic comprising:
(i) receiving logic executed by the processor for receiving, over a network from a user, a cell viability query comprising cell viability data associated with a testable culture of a patient's tumor;
(ii) extracting logic executed by the processor for extracting features of the cell viability data from the testable culture, said features comprising information associated with one or more targeted drugs;
(iii) generating logic executed by the processor for generating a probabilistic target inhibition map (PTIM) from the viability data that produces the lowest error in sensitivity prediction; wherein the computational algorithm involves (i) a selection of a set of targets that satisfies the biological constraint of increased inhibition of oncogenic targets increasing sensitivity, and (ii) generation of a probabilistic model based on the selected targets that produces high accuracy sensitivity prediction for unknown drugs with known target inhibition profile;
(iv) receiving logic executed by the processor for receiving secondary data from one or more secondary screens from the testable culture;
(v) comparing logic executed by the processor for comparing the PTIM of the cell viability data with the secondary data from one or more cancer screens from the testable culture, said comparison comprising creating a genomics and proteomics informed PTIM (GPI-PTIM);
(vi) designing logic executed by the processor for designing a designed combination therapy based on the GPI-PTIM, wherein the GPI-PTIM is configured to yield higher sensitivity with minimal target inhibition or avoiding resistance to drugs by targeting multiple pathways; and
(vi) communicating logic executed by the processor for communicating, over the network a designed combination therapy.

17. The system of claim 16, wherein the designing logic further comprises logic for considering the selection of a set of drugs from available drugs using approximation algorithms, wherein the combined toxicity of the set of drugs is restricted by an upper bound and the higher sensitivity is predicted to be synergistic.

18. The system of claim 16, further comprising treating the patient with the validated combination therapy.

19. The system of claim 16, wherein the combination therapy will be the designed combination therapy that demonstrates the best activity against the cancer in vitro and in vivo.

20. The system of claim 16, wherein the designing logic comprises considering the selection of a set of drugs from available drugs, wherein the combined toxicity of the set of drugs is restricted by an upper bound and the sensitivity of the combination drugs is predicted to be synergistic.
